(19) United States
(12) Patent Application Publication
(10) Pub. No.: US 2010/0061973 A1
(43) Pub. Date: Mar. 11, 2010

(54) Graded Expression of Snail as Marker of Cancer Development and DNA Damage-Based Diseases

(76) Inventors: Isidro Sanchez-Garcia, Salamanca (ES); Felipe Voces-Sanchez, Salamanca (ES); Pedro Antonio Perez-Mancera, Salamanca (ES)

Correspondence Address: MORRISON & FOERSTER LLP, 425 MARKET STREET, SAN FRANCISCO, CA, 94105-2482, US

(21) Appl. No.: 11/989,382

(22) PCT Filed: Jul. 28, 2006
(86) PCT No.: PCT/IB2006/002571

§ 371 Date: Jul. 17, 2009

(30) Foreign Application Priority Data
Jul. 28, 2005 (EP) 05076753.2

(51) Int. Cl. A61K 39/395; A01K 67/027; G01N 33/574; G01N 33/50; C12N 5/10; C12Q 1/02; C07H 21/02; C07K 16/00; C07K 14/00; C12N 9/00; A61K 31/7088; A61K 38/43; A61K 38/00

(52) U.S. Cl. 424/130.1; 800/13; 800/18; 800/10; 800/3; 435/325; 435/29; 536/23.1; 536/24.5; 530/387.1; 530/350; 435/183; 514/44 A; 514/44 R; 424/94.1; 514/2

(57) ABSTRACT

The invention relates to the graded expression level of SNAIL gene or its expression products, as a marker of the capacity of epithelial and mesenchymal tumours and/or cancers for disseminating to other tissues or organs. The invention further relates to a transgenic non-human mammal comprising in its genome a transgene that comprises a nucleic acid sequence encoding the SNAIL protein, and the use of SNAIL as a marker of epithelial and mesenchymal tumours and/or cancers as well as in DNA damage-based diseases In addition, the invention relates to the use of SNAIL as a therapeutic and diagnostic target for said pathologies.

### FIELD OF THE INVENTION

The invention relates, in general, to markers of cancer development; in particular, with the graded expression level of SNAIL gene or its expression products, as a marker of the capacity of epithelial and mesenchymal tumours and/or cancers for disseminating to other tissues or organs. The invention further relates to the use of SNAIL as a marker of epithelial and mesenchymal tumours and/or cancers as well as in DNA damage-based diseases. The invention further relates to the use of SNAIL as a therapeutic and diagnostic target for said pathologies. In addition, the invention relates to transgenic non-human animals that express SNAIL in a controllable fashion.

### BACKGROUND OF THE INVENTION

The SNAIL family of zinc-finger transcription factors occupies a central role for mesoderm formation in several organisms from flies to mammals. The first member of the SNAIL family, SNAIL, was described in Drosophila melanogaster, where it was shown to be essential for the formation of mesoderm. The transfection of SNAIL in mammalian epithelial cells and the phenotype of the SNAIL-mutant mice, where is essential for gastrulation, confirmed this function. The SNAIL protein is a transcriptional repressor which acts to maintain proper germ layer boundaries by repressing the expression within the mesoderm of regulatory genes involved in ectodermal development. Two mouse homologues of SNAIL, termed SNA and SLUG, have been cloned. It has been previously demonstrated that mice homozygous for a null mutation of the SLUG gene are viable, although they exhibit postnatal growth deficiency.

In addition to their roles in pattern formation and specification of mesoderm, some members of the SNAIL superfamily have been implicated in cell survival. In vitro studies have shown that SNAIL attenuates the cell cycle and confers resistance to cell death induced by the withdrawal of survival factors (Vega et al, 2004) or by DNA damage (Kajita et al, 2004). Cells expressing SNAIL or SLUG were protected from apoptosis induced by DNA-damaging agents, such as chemotherapeutic agents. Analysis of apoptotic pathways revealed that ectopic expression of SNAIL leads to downregulation of multiple genes with known roles in programmed cell death. The resistance to cell death conferred by SNAIL provides a selective advantage to cells to separate from the primary site and migrate. SNAIL family of genes are evolutionarily conserved, and studies have implicated SNAIL family proteins in the regulation of epithelial-mesenchymal transitions (EMT) in tissue culture systems and in both vertebrate and invertebrate embryos.

Epithelial-mesenchymal transition is the mechanism by which epithelial cells can dissociate from the epithelium and migrate. As such, EMT is fundamental to both normal development and the progression of epithelial tumours. Thus, SNAIL expression is able to trigger EMT and is being increasingly recognised as an alteration in cancer. Approximately 90% of cancer deaths result from the local invasion and distant metastasis of tumour cells. One important insight came from the discovery that the increased motility and invasiveness of cancer cells is reminiscent of the EMT that occurs during embryonic development. In EMT epithelial cells acquire fibroblast-like properties and show reduced intercellular adhesion and increased motility. This process is associated with the functional loss of E-cadherin. Stable expression of SNAIL in prototypic epithelial cell system of MDCK cells induces a complete epithelial to mesenchymal transition and these cells overexpressing SNAIL exhibit tumorigenic properties when injected in nude mice. The involvement of SNAIL in tumour progression is also supported by its expression in invasive carcinoma cell lines, and by the expression of SNAIL in the invasive cells of tumours induced in the skin of mice and in biopsies from patients with ductal breast carcinomas, gastric cancer, hepatocellular carcinomas (Sugimachi et al., 2003), and synovial sarcomas (Saito et al, 2004). Thus, SNAIL overexpression appears to be correlated with invasive growth potential in human cancer and it could therefore be of importance to cell fate selection by genotoxic anticancer agents.

### SUMMARY OF THE INVENTION

One aspect of the invention is based on the finding that the differential expression level of SNAIL gene is associated with a different effect on the development of epithelial and mesenchymal tumours and/or cancers. Inventors have observed that above a determined expression level of SNAIL (threshold level) the invasive and/or metastatic capacity of said epithelial and mesenchymal tumours and/or cancers increases, whereas SNAIL expression levels below this threshold level induces a tumorigenic but not migratory phenotype of these tumours.

Transformation depends upon genetic changes that allow undifferentiated cells to grow outside their normal environment. Evidence is provided herein that under certain circumstances, SNAIL expression facilitates cell migration. Furthermore, “increased” SNAIL expression induces cancer in mice with high frequency.

In particular; the inventors have found that in the normal course of events, SNAIL expression would be down-regulated by DNA damage in a P53-independent fashion. Both in vivo and in vitro, SNAIL expression is modulated in response to DNA damage in a p53-independent manner. However, when SNAIL is released from this regulated expression, it causes dissemination of cancers. The present inventors' results connect DNA damage with the requirement of a critical level of an EMT regulator for cancer development and it seems likely that failure to regulate SNAIL explains why the animal models described herein develop cancer. These findings further indicate that overexpression of Snail by human tumours could be of importance to cell fate selection by genotoxic anticancer agents. Indeed, human cancers that overexpress SNAIL may have a survival advantage to genotoxic and potentially other forms of stress by exploiting physiologic mechanisms that evolved for the EMT, raising the possibility of strategies based on SNAIL for the prevention and/or treatment of human cancer. Accordingly, a transgenic model in which SNAIL is expressed in a controllable fashion is of immense utility since this model recapitulates disseminated human cancers with high fidelity.

The invention is also based on the finding that SNAIL is expressed in epithelial and mesenchymal tumours and/or cancers as well as in DNA damage-based diseases. Consequently, SNAIL can be used as a marker for said pathologies.

In addition, inhibiting or reducing the expression of SNAIL could be used for preventing and/or treating epithelial and mesenchymal tumours and/or cancers as well as DNA damage-based diseases. Consequently, SNAIL can be used as a target for screening compounds for use in the prevention and/or treatment of said pathologies.

In the present invention, in order to further investigate the function of SNAIL during cancer development, mice harbouring a tetracycline-repressible SNAIL transgene were generated. These mice did not exhibit morphological defects at birth but did develop cancers similar to those associated with SNAIL expression in humans. These defects were not corrected by suppression of the SNAIL transgene. It has been found that Combi-tTA-SNAIL mouse embryonic fibroblasts (MEFs) and mice expressed SNAIL at levels considerably lower than those of endogenous counterparts. It is further shown that Combi-tTA-SNAIL does not confer a migratory advantage, although it does induce tumour formation. Combi-tTA-SNAIL expression results in increased radioprotection in vivo. SNAIL expression is repressed following DNA damage in a p53-independent manner. Thus, it seems likely that failure to regulate SNAIL expression explains why Combi-tTA-SNAIL mice develop cancer. These results suggest that tightly graded increase of SNAIL can induce cancer.

### BRIEF DESCRIPTION OF THE FIGURES

FIG. 1 shows the Combi-tTA-SNAIL: transgene construct, expression, and effect of SNAIL on the survival of Ba/F3 cells deprived of growth factor. FIG. 1A shows a schematic representation of the cassette used to replace the tetO-luciferase cassette of the original Combi-tTA-Vector described by Schultze et al. (1996) FIG. 1B shows a schematic representation of the Combi-tTA-SNAIL vector used in this invention, as obtained by modification of the original Combi-tTA-Vector described by Schultze et al. (1996) using the cassette shown in FIG. 1A. FIG. 1C shows an analysis of tetracycline-dependent SNAIL expression by RT-PCR in Ba/F3 cells for Combi-tTA-SNAIL (−tet, +tet in the medium). The PCR products were transferred to a nylon membrane and analyzed by hybridization with a specific probe for SNAIL. β-actin was used to check cDNA integrity and loading. FIG. 1D shows the survival of Ba/F3 cells expressing SNAIL in the absence of IL-3. Cells growing exponentially in IL-3 supplemented media were adjusted to 5×10⁵ cells/ml on day 0, and cultured after removal of IL-3. The cell number of viable cells is shown for SNAIL-transfected Ba/F3 cells grown in the absence of IL-3. FIG. 1E shows that cell death is accompanied by nucleosome laddering after IL-3 deprivation. Low molecular weight DNA was isolated 24 hours after IL-3 deprivation from Ba/F3-Combi-tTA-SNAIL grown in the absence of IL-3 and doxycycline (−tet) (lane 1), and Ba/F3-Combi-tTA-SNAIL grown in the absence of IL-3 and with doxycycline (+tet) (lane 2). The time of treatment with doxycycline was 48 hours. DNA was end-labelled, resolved by electrophoresis in a 2% agarose gel, and visualised by autoradiography. FIG. 1F shows, for the example of BCR-ABL^{p190} as the transgene, that tightly regulated control of the transgene by the tetracyclin derivative doxycyclin (Dox) was not possible using the original original Combi-tTA-Vector described by Schultze et al. (1996), but was rather only possible after modification of said original vector. The modification of the original vector, as carried out for SNAIL as the transgene, is described in FIGS. 1A and 1B.

In FIG. 2 it is shown the transgene expression in Combi-tTA-SNAIL mice. FIG. 2A shows the identification of transgenic mice by Southern analysis of tail snip DNA after EcoRI digestion. We used the cDNA for mouse SNAIL for detection of the transgene. In FIG. 2B the expression of the transgene was demonstrated by RT-PCR. Expression of Combi-tTA-SNAIL and endogenous SNAIL was analyzed by RT-PCR in tissues derived of Combi-tTA-SNAIL and control mice. β-actin was used to check cDNA integrity and loading.

FIG. 3 illustrates the deficient T-cell development in thymus of Combi-tTA-SNAIL mice. Representative analysis of the cells present in the thymus of these mice is shown. Cells isolated from a wild-type (control), and a Combi-tTA-SNAIL mouse were stained with the monoclonal antibodies and analyzed by flow cytometry. The percentage of cells is indicated.

FIG. 4 shows hematopoietic cancers in Combi-tTA-SNAIL mice. FIG. 4A illustrates the phenotypic characteristics of leukemias of Combi-tTA-SNAIL mice. Cells from bone marrow (BM), peripheral blood (pb) and spleen of Combi-tTA-SNAIL mice were analyzed by flow cytometry. Cells were identified with combinations of specific antibodies. Cells (10,000) were collected for each sample and dead cells were excluded from analysis by propidium iodide staining. In FIG. 4B are shown the hematoxylin/eosin stained sections of the spleen of wild-type and Combi-tTA-SNAIL mice. The spleen from Combi-tTA-SNAIL mice shows the effacement of the normal spleen architecture. FIGS. 4C, 4D, 4E show the histological appearance of tissues in leukaemic Combi-tTA-SNAIL mice. Leukaemic cells disobey the social order of organ boundaries and migrate as individual cells giving metastasis to different regions (liver, kidney and lung).

In FIG. 5 it is represented the carcinoma development in Combi-tTA-SNAIL mice. Histological analysis of lung (A), testis (B) and liver (C) of wild-type and Combi-tTA-SNAIL mice. Representative matched tissue sections from wild-type and Combi-tTA-SNAIL mice were stained with Hematoxylin/Eosin. The histological sections of Combi-tTA-SNAIL lung show the presence of an adenocarcinoma (A). The histological section of Combi-tTA-SNAIL testis shows the presence of a hyperplasia of germ cells (B). The histological sections of Combi-tTA-SNAIL liver show the presence of a hepatocarcinoma (C).

In FIG. 6 it is presented the cancer development in CombiTA-SNAIL mice after suppression of SNAIL expression by tetracycline treatment. FIG. 6A shows an analysis of tetracycline-dependent SNAIL expression in peripheral blood of mice transgenic for Combi-tTA-SNAIL (−tet, +tet in water) by RT-PCR. Actin was used to check cDNA integrity and loading. FIG. 6B there are shown the representative flow cytometry phenotypic characteristics of cells from thymus, bone marrow (BM) and peripheral blood (pb) of Combi-tTA-SNAIL mice after suppression of SNAIL expression by tetracycline treatment (4 gr/L) for 4 weeks. Cells were stained with the monoclonal antibodies and analyzed by flow cytometry. The percentage of cells is indicated. FIG. 6C illustrates representative Hematoxylin/Eosin stained sections of tissues in Combi-tTA-SNAIL mice after suppression of SNAIL expression by tetracycline treatment (4 gr/L) for 4 weeks.

In FIG. 7 it is shown that Combi-tTA-SNAIL mice have a graded increase of Combi-tTA-SNAIL expression. FIG. 7A represents quantitative real-time RT-PCR analysis of spleen and MEF RNA samples showed that Combi-tTA-SNAIL expression was increased to ˜20% of endogenous SNAIL level in transgenic mice. Combi-tTA-SNAIL and endogenous SNAIL transcript numbers are shown as a percentage of β-actin transcripts. In FIG. 7B the expression of Combi-tTA-SNAIL was analyzed by RT-PCR in lung carcinoma (lane 1) and hepatocarcinoma (lane 3) tissues derived of Combi-tTA-SNAIL mice. Actin was used to check cDNA integrity and loading.

FIG. 8 shows that Combi-tTA-SNAIL expression in MEFs does not induce a migratory phenotype. The motility/migratory behaviour of control-MEFs (a, b, and c) and Combi-tTA-SNAIL-MEFs (d, e, and f) was analyzed in an in vitro wound model. Confluent cultures were gently scratched with a pipette tip to produce a wound. Photographs of the cultures were taken immediately after the incision (a, d) and after 9 h (b, e) and 15 h (c, f) in culture.

In FIG. 9 it is shown the effect of irradiation on survival of Combi-tTA-SNAIL mice. In FIG. 9A it is shown that Combi-tTA-SNAIL (30 animals) and control mice (30 animals) were irradiated at 950 rads to determine their survival after DNA-damage. The radiation dose was given as a split dose of equal intensity, 4 h apart. FIG. 9B illustrates the levels of p53 protein in Combi-tTA-SNAIL and control BM cells after γ-irradiation p53 protein was detected by Western-blotting. Actin was used as a loading control. The time points are in hours.

FIG. 10 represents the identification of SNAIL as a DNA-damage transcriptionally regulated gene. In FIG. 10A it is shown a Northern blot analysis of SNAIL expression in MEFs from different genotypes following DNA damage. RNAs were prepared from cells treated/not treated with doxorubicin (+/− dox). After hybridization with a SNAIL cDNA probe, the same blot was rehybridized with BclxL and p21 probes as positive controls. Loading was monitored with ARPP-PO. In FIG. 10B it is illustrated that P53 does not transactivate the SNAIL promoter. Luciferase reporter assays demonstrate independent responsiveness of the human SNAIL reporter to P53. The number shown at the left of the reporter constructs denotes the 5′-boundaries (bp upstream of the initiation site). FIG. 10C shows the in vivo regulation of SNAIL expression in response to DNA damage. In spleen of mice six hours after 5 Gy of γ-radiation, SNAIL expression is reduced in both the wild-type and the p53−/− spleen tissues. Northern blots were hybridized with SNAIL, and ARPP-PO (U, untreated).

FIG. 11 shows large thymic lymphomas that are developed by Combi-tTA-SNAIL-p53−/− mice at an age of 2-3 months and infiltrate the lung, the heart, the mediastinal space.

FIG. 12 shows micrographs of histological preparations of lymphoma, lung tumour and sebaceoma samples.

### DETAILED DESCRIPTION OF THE INVENTION

### Definitions

In order to facilitate the understanding of the instant description, the meaning of some terms and expressions in the context of the invention are explained below.

The term “subject” as used in this description refers to members of mammal species, and includes, but is not limited to, domestic animals, rodent, primates and humans; the subject is preferably a human being, male or female, of any age or race.

The term “sample”, as used herein, can be any biological sample from a subject, such as a liquid sample, for example, blood, serum, etc., or a solid sample, such as a tissue sample, etc. The sample can be obtained by any conventional method, including surgical resection in case of solid samples. The sample can be obtained from a subject previously diagnosed, or not diagnosed, with an epithelial or mesenchymal tumour, or from a subject previously diagnosed, or not diagnosed, with a DNA damage-based disease; or also from a subject undergoing treatment, or who has been previously treated, for any of said pathologies. In an embodiment, the sample is a liquid or solid biological sample from an epithelial or mesenchymal tumour.

The term “epithelial cancer”, as used herein, refers to a cancer of which tumour cells are the cells that line the internal and external surfaces of the body. The term “mesenchymal cancer”, as used herein, refers to a cancer which tumour cells develop into connective tissue, blood vessels and lymphatic tissue. Illustrative, non-limitative examples of said epithelial or mesenchymal cancers include lymphomas, leukaemias, sarcomas and carcinomas, such as, for example, chronic myeloid leukaemia, B-cell acute lymphoblastic leukaemia, T-cell acute lymphoblastic leukaemia, acute myeloid leukaemia, chronic myeloid leukaemia, lymphoproliferative syndromes, multiple myeloma, liposarcoma, and Ewing sarcoma (Best and Taylor. Bases fisiológicas de la patología médica. Madrid: Editorial Médica Panamericana, 12th ed., 1993).

The term “DNA damage-based disease” refers to a disease based on DNA damage in a subject which can occur from interactions with radiation, chemicals that form adducts with the bases of DNA, structural impediments to transcription and replication, genetic predisposition and spontaneous loss of bases. Illustrative, non-limitative examples of said diseases include xeroderma pigmentosusm, cockayne syndrome, trichothiodystrophy, bloom syndrome, Werner syndrome, Rothmund-Thomson syndrome, ataxia telangiectasia, Nijmegen breakage syndrome, Fanconi anemia, hereditary nonpolyposis colorectal cancer, etc. (Robb E Moses, 2001. DNA damage processing defects and disease. Annu. Rev. Genomics Hum. Genet. 2:41-68).

The term “gene” refers to a molecular chain of deoxyribonucleotides encoding a protein.

The term “DNA” refers to deoxyribonucleic acid. A DNA sequence is a deoxyribonucleotide sequence.

The term “cDNA” refers to a nucleotide sequence complementary of a mRNA sequence.

The term “RNA” refers to ribonucleic acid. An RNA sequence is a ribonucleotide sequence.

The term “mRNA” refers to messenger ribonucleic acid, which is the fraction of total RNA which is translated into proteins.

The term “protein” refers to a molecular chain of amino acids with biological activity.

The term “SNAIL protein” refers to a member of the SNAIL family of zinc-finger transcription factors which is a transcriptional repressor that acts to maintain proper germ layer boundaries by repressing the expression within the mesoderm of regulatory genes involved in ectodermal development. The amino acid sequence of the human SNAIL protein is known (see, for example, NCBI, Accession number AAH12910).

The term “SNAIL gene” refers to the gene coding for the SNAIL protein. The nucleotide sequence of the human SNAIL gene is known (see, for example, NCBI, Accession number BC012910) and this is the preferred gene for use in aspects of the invention referred to herein.

The term “transcription product of SNAIL gene” refers to the mRNA of SNAIL gene.

The term “translation product of SNAIL gene” refers to SNAIL protein. Again, the human SNAIL protein is preferred.

The term “antibody” refers to a glycoprotein exhibiting specific binding activity to a particular protein, which is called “antigen”. The term “antibody” comprises monoclonal antibodies, polyclonal antibodies, either intact or fragments thereof, recombinant antibodies, etc., and includes human, humanized and non-human origin antibodies. “Monoclonal antibodies” are homogenous populations of highly specific antibodies directed against a single site or antigenic “determinant”. “Polyclonal antibodies” include heterogeneous populations of antibodies directed against different antigenic determinants.

The term “epitope”, as it is used in the present invention, refers to an antigenic determinant of a protein, which is the amino acid sequence of the protein recognized by a specific antibody.

### 1. Graded Expression of SNAIL Gene or SNAIL Protein as Marker of Cancer Development

As previously mentioned, the invention is based on the finding that the differential expression level of SNAIL is associated with a different effect on the development of epithelial and mesenchymal tumours and/or cancers. In particular, the inventors have observed that above a determined expression level of SNAIL (threshold level) the invasive and/or metastatic capacity of said epithelial and mesenchymal tumours and/or cancers increases, whereas SNAIL expression levels below said threshold level induces a tumorigenic but not migratory phenotype of said tumours.

### 1.1 Invasive and Metastatic Capacity of Epithelial or Mesenchymal Tumour Cells

In an aspect, the invention refers to the discovery that differential expression of the SNAIL gene or SNAIL protein is related with the invasive and metastatic capacity of epithelial or mesenchymal tumour cells in a subject suffering from epithelial or mesenchymal cancer. SNAIL expression that has been unhinged from its normal regulation mechanisms has been identified herein as a marker of dissemination capability. Thus, the expression or repression of the SNAIL gene, its expression products (including both transcription and translation products, i.e, mRNA or SNAIL protein) as well as the expression or repression of products related with the regulation of said gene, or with the elimination or degradation of its expression products, can be used to evaluate the risk of a subject suffering from epithelial or mesenchymal cancer, whose cancer cells are SNAIL+, to develop invasion, dissemination and/or metastasis. Therefore the SNAIL gene and its expression products (including both transcription and translation products, i.e, mRNA and SNAIL protein) are useful markers of the malignity of said epithelial or mesenchymal tumour cells and constitute very attractive targets for the treatment, prevention and/or diagnosis of epithelial or mesenchymal cancer.

Therefore, in an aspect, the invention relates to a method for determining the invasive, dissemination and/or metastatic capacity of an epithelial or mesenchymal tumour, which comprises:
(i) quantifying the level of SNAIL mRNA or the level of SNAIL protein expressed in a test sample obtained from said tumour, and
(ii) comparing said level to that of a control sample,
   wherein an increase in said level relative to that of the control sample, said increase being of at least 20% above the level of the control sample, is indicative of invasive and/or metastatic capacity of said tumour. The increase above the level of the control sample may be 30%, 40%, 70%, 100%, 150%, 200% or more.

In order to carry out the above mentioned method, a sample is obtained from the subject under study. Samples can be obtained from subjects previously diagnosed or not with epithelial or mesenchymal tumours and/or cancers, or from subjects who are receiving or have previously received therapy for treating said epithelial or mesenchymal tumours and/or cancers. In a particular embodiment, the sample is a biological sample from said epithelial or mesenchymal tumour. The samples can be obtained by conventional methods, e.g., extraction, surgical resection, biopsy, etc., by using methods well known to those of ordinary skill in the related medical arts. Methods for obtaining the sample from the biopsy include gross apportioning of a mass, or microdissection or other art-known cell-separation methods.

Because of the variability of the cell types in diseased-tissue biopsy material, and the variability in sensitivity of the diagnostic methods used, the sample size required for analysis may range from 1, 10, 50, 100, 200, 300, 500, 1,000, 5,000, 10,000, to 50,000 or more cells. The appropriate sample size may be determined based on the cellular composition and condition of the biopsy, and the standard preparative steps for this determination and subsequent isolation of the nucleic acid for use in the invention are well known to one of ordinary skill in the art. An example of this, although not intended to be limiting, is that in some instances a sample from the biopsy may be sufficient for assessment of RNA expression without amplification, but in other instances the lack of suitable cells in a small biopsy region may require use of RNA conversion and/or amplification methods or other methods to enhance resolution of the nucleic acid molecules. Such methods, which allow the use of limited biopsy materials, are well known to those of ordinary skill in the art and include, but are not limited to: direct RNA amplification, reverse transcription of RNA to cDNA, amplification of cDNA, or the generation of radio-labelled nucleic acids.

In a particular embodiment, with the aim of quantifying the level of SNAIL mRNA in a sample, the above defined method comprises a step for extracting the sample and obtaining the total RNA extract. This extract represents the working material for the next step. Total RNA extraction protocols are well known by a person skilled in the art (Chomczynski P. et al., Anal. Biochem., 1987, 162: 156; Chomczynski P., Biotechniques, 1993, 15: 532). Any conventional method can be used within the framework of the invention for determining the invasive and/or metastatic capacity of an epithelial or mesenchymal tumour, as long as the in vitro measurement of SNAIL gene transcribed mRNA or its complementary cDNA can be performed in samples taken from the subjects to be analyzed (test samples) and from control samples.

Once the sample has been obtained and the total RNA has been extracted, the quantification of the level of SNAIL mRNA can be carried out, in a particular embodiment, by quantifying the level of SNAIL mRNA or the level of the corresponding cDNA of the SNAIL mRNA.

In an example, detection and quantification of SNAIL mRNA is carried out by blotting the mRNA onto a nylon membrane by means of blotting techniques, such as, for example, Northern blot, and detecting it with specific probes of the SNAIL mRNA or of its cDNA.

In another example, the quantification of SNAIL mRNA can be achieved by a two-step method comprising a first step of amplification of the RNA, preferably mRNA, or amplification of the cDNA synthesized by reverse transcription (RT) from the SNAIL mRNA, and a second step of quantification of the amplification product of the SNAIL mRNA or its corresponding cDNA. One example of mRNA amplification consists in reverse transcribing the mRNA into cDNA, followed by the Polymerase Chain Reaction (PCR) using the appropriate oligonucleotide primers (U.S. Pat. No. 4,683,195, U.S. Pat. No. 4,683,202 and U.S. Pat. No. 4,965,188). Many methods for detecting and quantifying the PCR amplification products have been previously disclosed, any of which methods could be used in this invention. In a particular embodiment, the amplification and quantification of the SNAIL mRNA is carried out by means of real time quantitative RT-PCR (Q-PCR) and subsequent hybridization with a probe specific for SNAIL, optionally said probe being labelled with an appropriate tag, as for example a radioactively labelled probe (e.g., radioactive, chemiluminescent, or fluorescent tags such as fluorescein, Cye3-dUTP, or Cye5-dUTP), hybridizing target genes to the probes, and evaluating target-probe hybridization. A probe with a nucleic acid sequence that perfectly matches the target sequence will, in general, result in detection of a stronger reporter-molecule signal than will probes with less perfect matches.

Probes to be used are specific for SNAIL mRNA or its cDNA. Said probes can be easily designed by the skilled person in the art in view of the nucleotide sequence of SNAIL gene by using any suitable software. The nucleotide sequence of the human SNAIL gene is known (NCBI, Accession number BC012910). According to the invention, probes are selected from the group of nucleic acids including, but not limited to, DNA, genomic DNA (gDNA), cDNA and oligonucleotides; and may be natural or synthetic. Oligonucleotide probes preferably are 20 to 25-mer oligonucleotides whereas DNA/cDNA probes preferably are 500 to 5,000 bases in length; nevertheless, in both cases, other lengths may be used.

The final step of the above defined method consists in comparing the level (amount or concentration) of SNAIL mRNA or the level of its cDNA determined in a sample obtained from said epithelial or mesenchymal tumour from the subject under analysis, with the level of SNAIL mRNA or with the level of its cDNA determined in control samples, such as samples from control subjects, i.e., samples from healthy subjects or samples from subjects free from epithelial and/or mesenchymal tumours and/or cancers, (i.e., subjects without a clinical history of epithelial and/or mesenchymal tumours and/or cancers) or in previous samples from the same subject.

The quantification of said products (SNAIL mRNA or its cDNA) is indicative of the state of development of an epithelial or mesenchymal tumour in a subject suffering from an epithelial and/or mesenchymal cancer, in particular, of the invasive and/or metastatic capacity of an epithelial and/or mesenchymal tumour. In this way, an increase in the transcription products of the SNAIL gene (e.g., SNAIL mRNA), or its cDNA, relative to the level of the control sample, said increase being of at least 20% above the level of the control sample (or at least 30%, 40%, 70%, 100%, 150%, 200% or more), is indicative of invasive, dissemination and/or metastatic capacity of said epithelial and/or mesenchymal tumour cells.

In the event that the SNAIL protein is to be detected, the above defined method comprises a first step in which the protein extract of the sample is placed in contact with a composition of one or more specific antibodies against one or more epitopes of the SNAIL protein, and a second step, in which the complexes formed by the antibodies of the SNAIL protein are quantified.

There is a wide variety of immunological assays available for detecting and quantifying the formation of specific antigen-antibody complexes; numerous competitive and non-competitive protein binding assays have been previously disclosed, and a large number of these assays are available on the market. Therefore, the SNAIL protein can be quantified with antibodies such as, for example: monoclonal antibodies, polyclonal antibodies, either intact or recombinant fragments thereof, combined antibodies and Fab or scFv antibody fragments, specific against the SNAIL protein; these antibodies being human, humanized or of a non-human origin. The antibodies used in these assays may be marked or not; the unmarked antibodies can be used in agglutination assays; the marked antibodies can be used in a wide variety of assays. The marker molecules which can be used for marking the antibodies include radionucleotides, enzymes, fluorophores, chemiluminescent reagents, enzyme substrates or cofactors, enzyme inhibitors, particles, dyes and derivatives. There is a wide variety of well known assays which can be used in the present invention using unmarked antibodies (primary antibody) and marked antibodies (secondary antibody); included among these techniques are Western-blot, ELISA (Enzyme-Linked immunosorbent assay), RIA (Radioimmunoassay), competitive EIA (Competitive enzyme immunoassay), DAS-ELISA (Double antibody sandwich-ELISA), immunocytochemical and immunohistochemical techniques, techniques based on the use of protein biochips or microarrays including specific antibodies or assays based on colloidal precipitation in formats such as dipsticks. Other ways of detecting and quantifying the SNAIL protein include affinity chromatography techniques, ligand binding assays or lectin binding assays. The preferred immunoassay in the method of the invention is a double antibody sandwich ELISA assay. Any antibody or combination of antibodies specific against one or more epitopes of the SNAIL protein can be used in this immunoassay. As an example of one of the many possible formats of this assay, a monoclonal or a polyclonal antibody, or a fragment of this antibody, or a combination of antibodies, coating a solid phase are placed in contact with the sample to be analyzed and are incubated for a time and under conditions which are suitable for forming the antigen-antibody complexes. An indicator reagent comprising a monoclonal or polyclonal antibody, or a fragment of this antibody, or a combination of these antibodies, bound to a signal generating compound is incubated with the antigen-antibody complexes for a suitable time and under suitable conditions after washing under suitable conditions for eliminating the non-specific complexes. The presence of the SNAIL protein in the sample to be analyzed is detected and quantified, in the event that it exists, by measuring the generated signal. The amount of SNAIL protein present in the sample to be analyzed is proportional to that signal. In this way, an increase in the level of SNAIL protein in the test sample relative to the level of SNAIL protein in a control sample, said increase being of at least 20% above the level of SNAIL protein in the control sample (or at least 30%, 40%, 70%, 100%, 150%, 200% or more), is indicative of invasive and/or metastatic capacity of said epithelial or mesenchymal tumour cells.

### 1.2 Local Growth Capacity of Epithelial or Mesenchymal Tumour Cells

In other aspect, the invention refers to the discovery that the level of SNAIL gene or its expression products (both transcription and translation products, i.e., mRNA and protein) is related with the local growth capacity of epithelial or mesenchymal tumour cells in a subject suffering from epithelial or mesenchymal cancer. In particular, an increase in the level of SNAIL gene or its expression products relative to that of the control sample, said increase being less than 20% above the level of the control sample (or at least 30%, 40%, 70%, 100%, 150%, 200% or more), is indicative of local growth of said epithelial or mesenchymal tumours and/or cancers. Thus, the expression or repression of the SNAIL gene, its expression products as well as the expression or repression of products related with the regulation of said gene, or with the elimination or degradation of its expression products, can be used to evaluate the predisposition of epithelial or mesenchymal tumour cells, in a subject suffering from epithelial or mesenchymal cancer, to grow locally. Therefore the SNAIL gene and its transcription products, and the products related with the regulation of said gene or protein or with the elimination or degradation of its expression products (including both transcription and translation products, i.e, mRNA or SNAIL protein) are useful markers of the capacity of said epithelial or mesenchymal tumour cells of locally growing and constitute very attractive targets for the treatment, prevention and/or diagnosis of epithelial or mesenchymal cancer.

Therefore, in an aspect, the invention relates to a method for determining the local growth capacity of an epithelial or mesenchymal tumour comprising:
(i) quantifying the level of SNAIL mRNA or the level of SNAIL protein in a test sample obtained from said tumour, and
(ii) comparing said level to that of a control sample,
   wherein an increase in said level relative to that of the control sample, said increase being less than 20% above the level of the control sample, is indicative of local growth of said tumour. The increase above the level of the control sample may be at least 30%, 40%, 70%, 100%, 150%, 200% or more.

As used herein, the term “local growth capacity”, opposite to invasive or metastatic capacity, refers to the capacity of a tumour of growing in the tissue or organ wherein uncontrolled division of tumour cells began; thus, said term can be applied, for example, to tumour cells which have not developed so far invasive and/or metastatic capacity, i.e., the ability of said cells to invade other tissues, either by direct growth into adjacent tissue (invasion) or by migration of cells to distant sites (metastasis), and, consequently, they grow locally in said tissue or organ.

In order to carry out the method for determining the local growth capacity of an epithelial or mesenchymal tumour, a sample from the subject under study has to be obtained. The particulars of the sample to be used in working this method are like those of the samples used in working the previously disclosed method for determining the invasive and/or metastatic capacity of an epithelial or mesenchymal tumour.

The quantification of the level of SNAIL mRNA or the level of SNAIL protein can be carried out by any of the techniques previously disclosed in connection with the method for determining the invasive and/or metastatic capacity of an epithelial or mesenchymal tumour. Subsequently, the level of SNAIL mRNA or the level of SNAIL protein quantified in the sample of the subject under study (test sample) is compared with the level of SNAIL mRNA or with the level of SNAIL protein in a control sample. In this case, an increase in said SNAIL mRNA level or SNAIL protein level relative to that of the control sample, said increase being less than 20% above the level of the control sample, is indicative of said epithelial or mesenchymal tumour of being capable of locally growing.

SNAIL as a Marker of Epithelial or Mesenchymal Tumours and/or Cancers or DNA Damage-Based Diseases

The invention is also based on the finding that SNAIL gene, or its expression products (including both transcription and translation products, i.e, SNAIL mRNA and SNAIL protein), are expressed in epithelial and mesenchymal tumours and/or cancers as well as in DNA damage-based diseases. Accordingly, the detection of SNAIL gene, or its expression products in a sample can be used in the diagnosis or prognosis of epithelial tumours, mesenchymal tumours and/or cancers or DNA damage-based diseases. In fact, the detection of SNAIL gene, or its expression products, in a sample is indicative of epithelial tumours, mesenchymal tumours or DNA damage-based diseases, or a greater risk or predisposition of the subject to develop epithelial tumours, mesenchymal tumours and/or cancers or DNA damage-based diseases. Therefore, the above mentioned finding can be used in one or more of the following methods: diagnostic assays, prognostic assays, monitoring clinical trials and screening assays as further described herein.

Therefore, in other aspect, the invention refers to an in vitro method for diagnosing the presence of a condition in a subject, said condition being selected from an epithelial tumour, a mesenchymal tumour and a DNA damage-based disease, or to determine the stage or severity of said condition in a subject, or to determine the predisposition of a subject to develop said condition, or to monitor the effect of the therapy administered to a subject with said condition, which comprises:
(i) determining the presence of a diagnostic marker in a sample from said subject, and
(ii) comparing the presence of said diagnostic marker with its absence in a control sample, wherein its presence is indicative of the presence of an epithelial or mesenchymal tumour or a DNA damage-based disease,
   wherein said diagnostic marker is SNAIL mRNA or SNAIL protein.

The detection of SNAIL mRNA or SNAIL protein, can be carried out by any of the techniques previously disclosed in connection with the method for determining the invasive and/or metastatic capacity of an epithelial or mesenchymal tumour. Subsequently, the detection of said products, e.g., SNAIL mRNA or SNAIL protein in the sample of the subject under study (test sample) is compared with its absence in a control sample, the presence of said products being indicative of the presence (diagnosis) of a condition selected from an epithelial tumour, a mesenchymal tumour and a DNA damage-based disease, in a subject under study, or of the predisposition of a subject to develop said condition. Thus, this method can also be used for monitoring the effect of the therapy administered to a subject with said condition and, if necessary, to select a further therapy.

### Transgenic Non-Human Mammal

In another aspect, the invention provides a transgenic non-human mammal, hereinafter referred to as the transgenic non-human mammal of the invention. Herein, a non-human mammal that is termed “transgenic” comprises a transgene in its genome. According to the invention, said transgene comprises a nucleic acid sequence encoding the SNAIL protein (i.e., said nucleic acid comprises the SNAIL gene), the expression of said transgene being exogenously regulated by an effector substance. Preferably the SNAIL protein is the human SNAIL protein (see, for example, NCBI, Accession number AAH12910), encoded by the human SNAIL gene (see, for example, NCBI, Accession number BC012910), although other forms, such as the murine form, may also be of some utility.

In certain embodiments of the invention, it may be appropriate to use as the transgene a sequence encoding only a portion of the SNAIL protein, such as a fragment, or a variant of the SNAIL protein. By “fragments” we mean any portion of the full length SNAIL protein, including, for example, 10%, 20%, 30%, 40%, 50%, 60%, 70%, 80%, 90%, 95% or more of the full length sequence. For example, a fragment may include a specific domain or combination of domains within the protein structure. By “variants”, we mean any variant of the SNAIL protein, such as, for example, a mutant form comprising one or multiple (e.g. 2, 3, 4, 5, 6, 7, 8, 9, 10 or more) insertions, deletions, substitutions and so on.

The transgenic non-human mammal provided by this invention possesses, as a result, a genotype that confers a greater tendency to develop a tumour selected from an epithelial tumour and a mesenchymal tumour, and/or DNA damage-based diseases and/or disseminated cancer when compared to the non-transgenic mammal. Examples of cancers that are generated in transgenic models of the type disclosed herein include those of both mesenchymal and epithelial origin. Specific examples are given in Table and include acute leukaemias, lymphomas, lung carcinomas, germ cell hyperplasias, hepatocarcinomas, hematopoietic neoplasias and acute myeloid leukaemias.

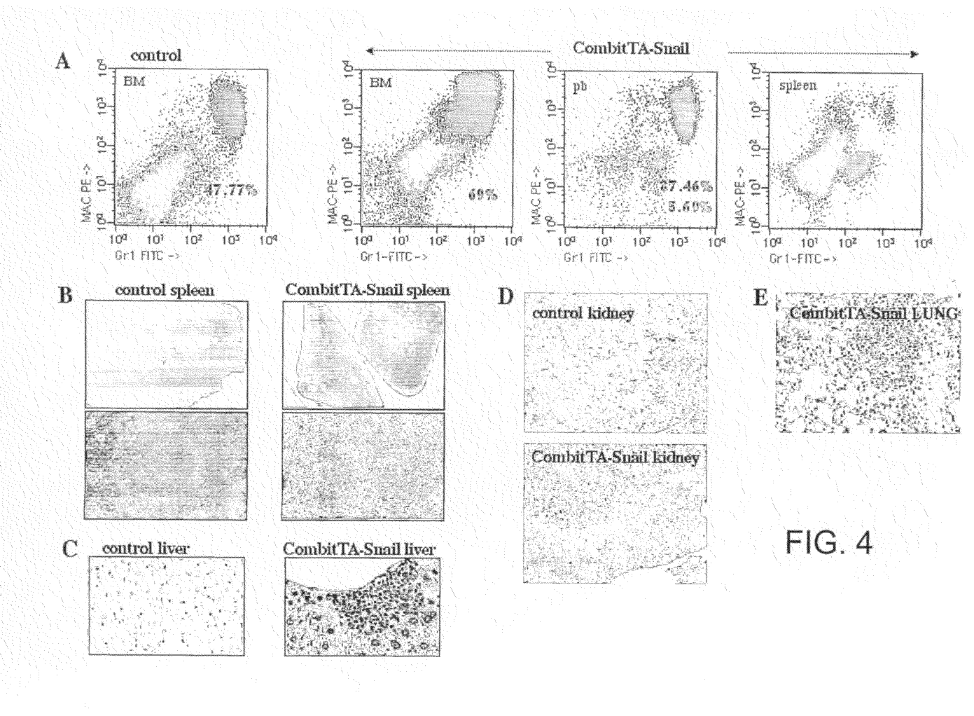

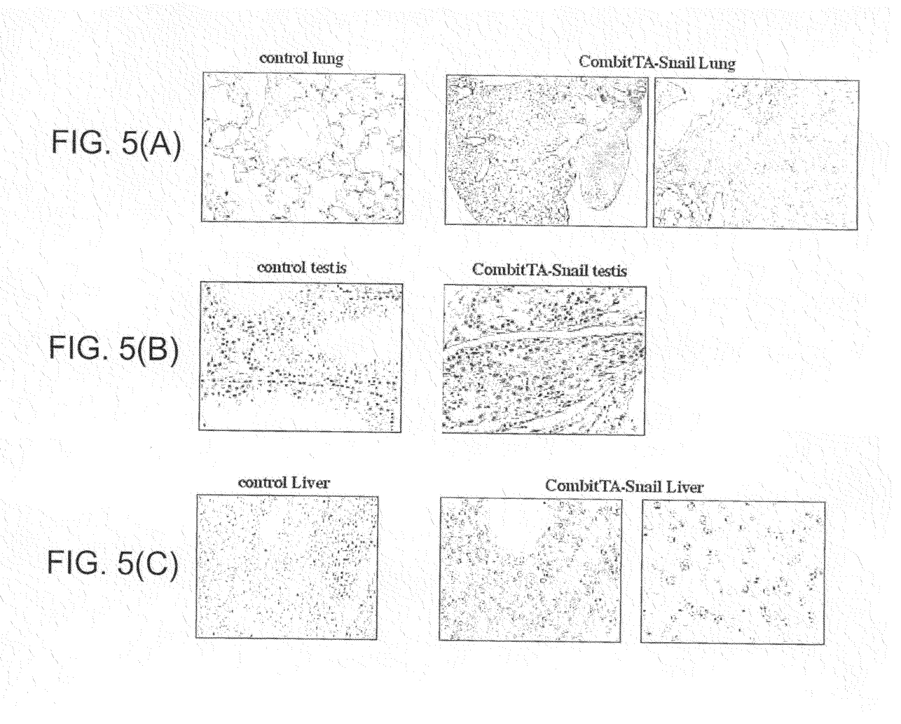

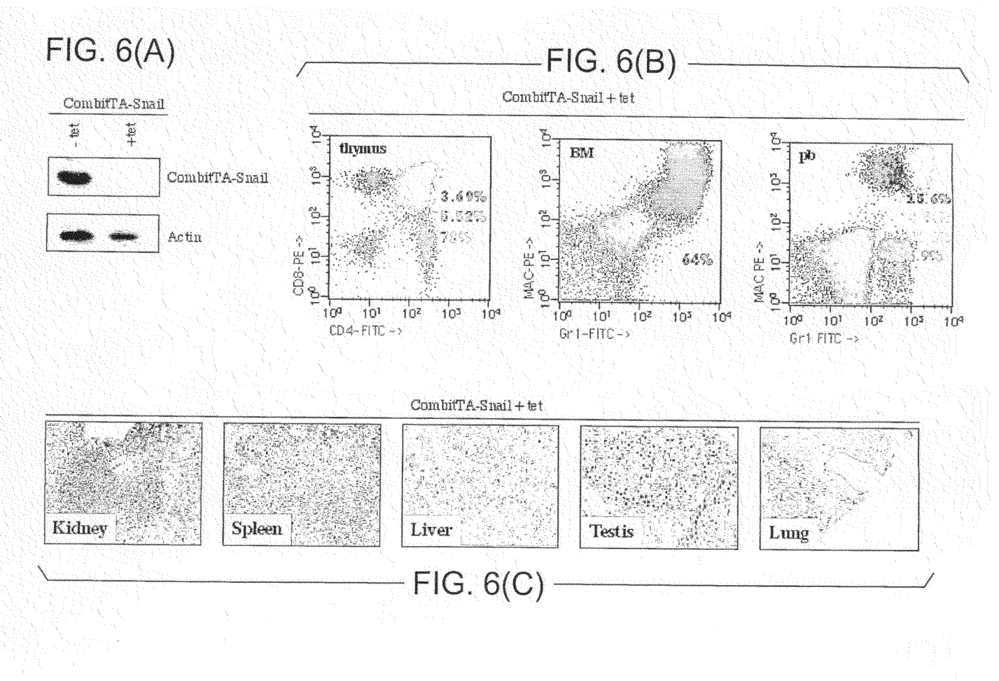

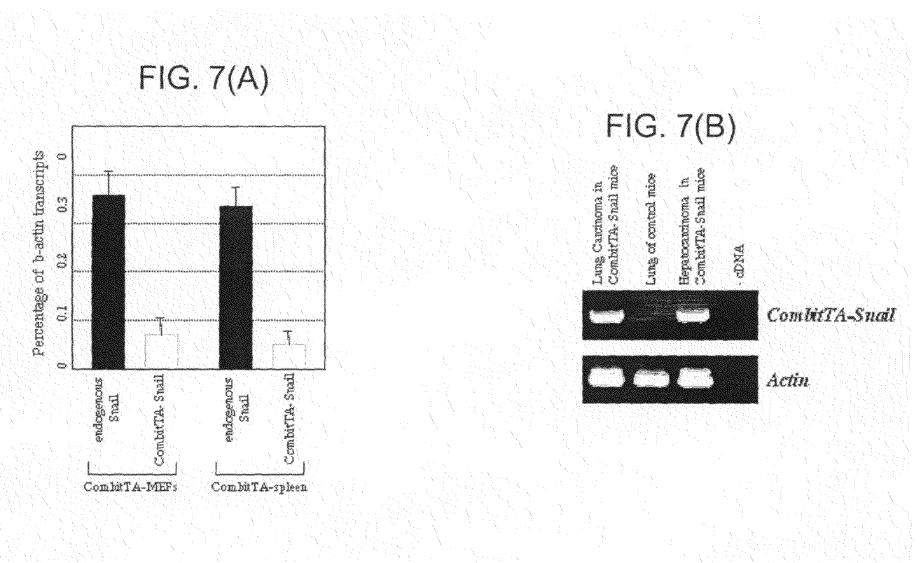

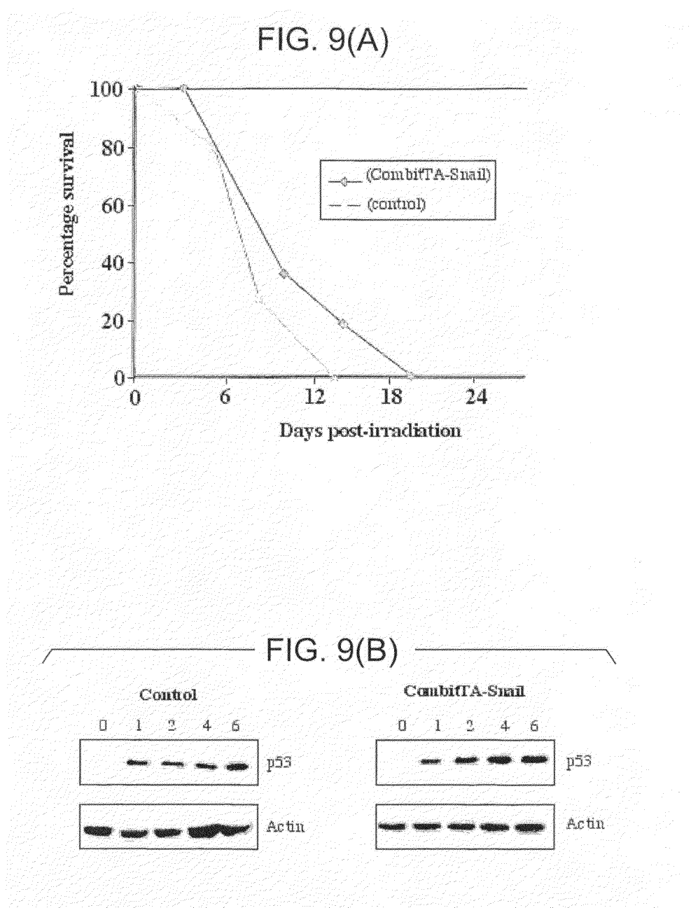

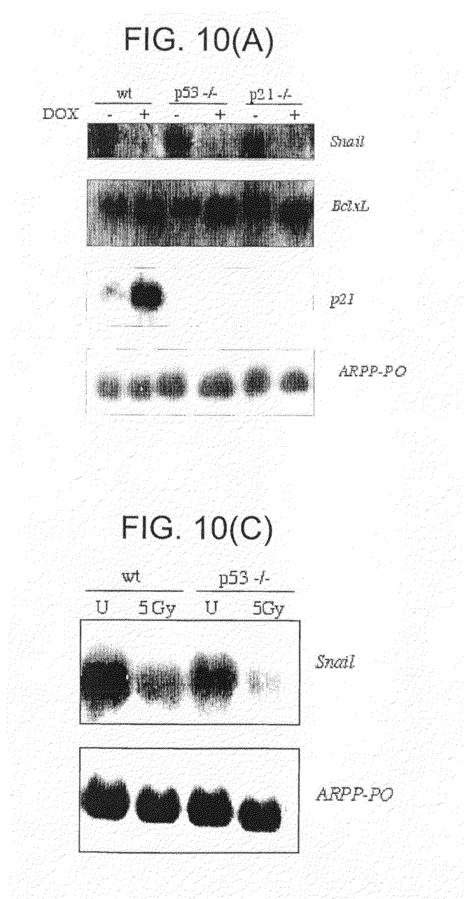

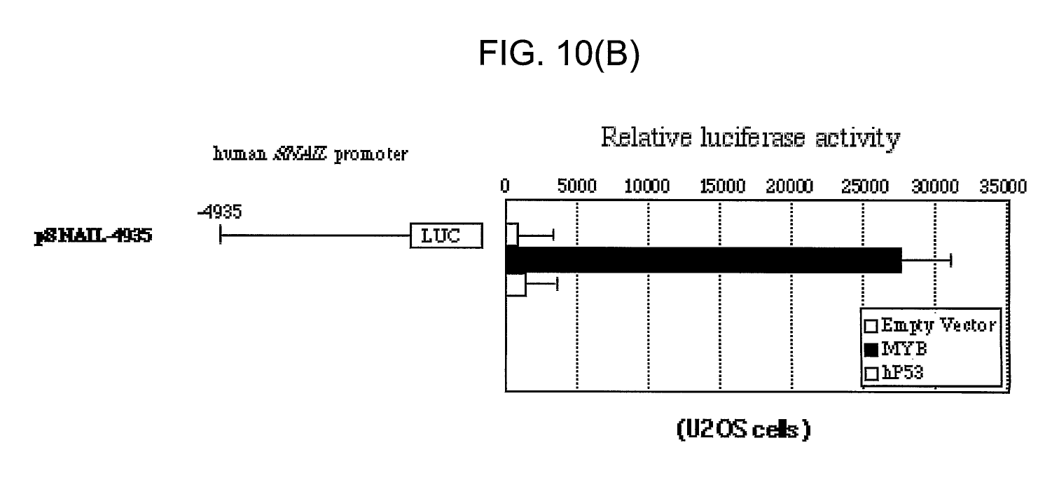

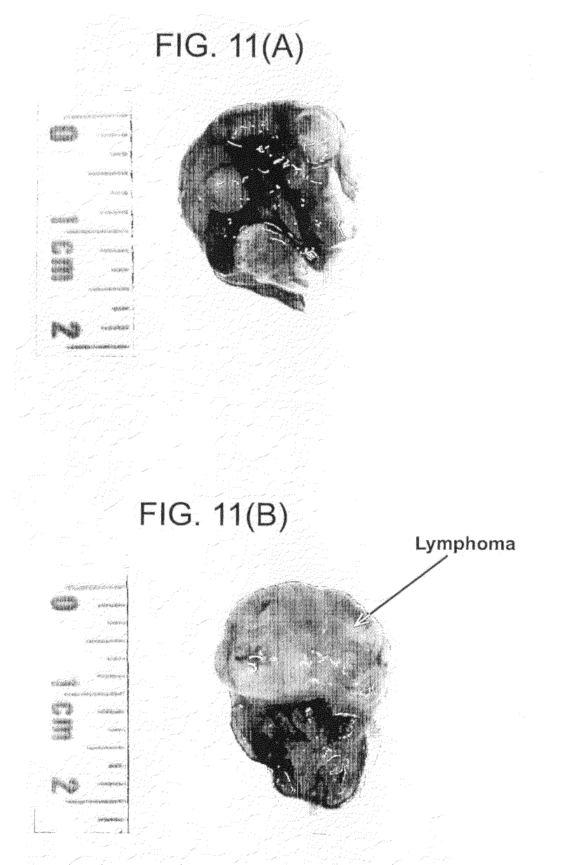

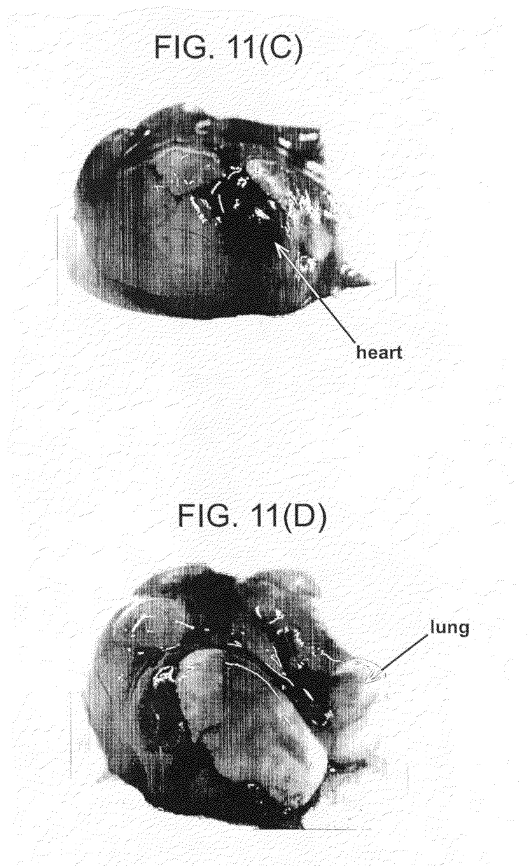

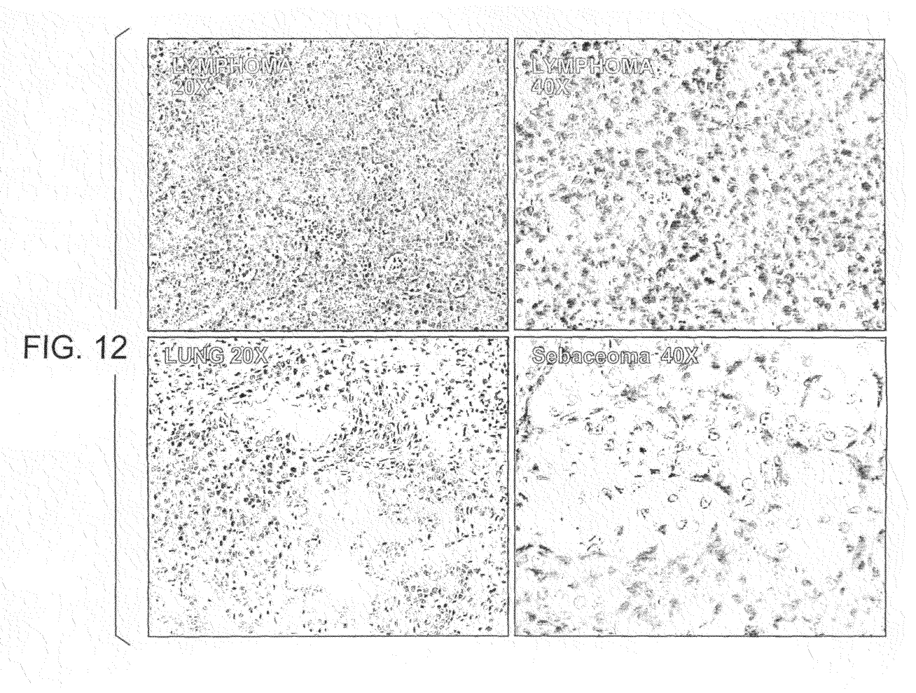

For example, all Combi-tTA-Snail mice generated herein (see examples) became unwell from approximately 5-7 months of age onward (Table I) with clinical manifestations that included decreased physical activity, tachypnea, pilo-erection, shivering, and sustained weight loss, prior to sacrifice. The cancers were from both mesenchymal and epithelial origin (Table I). The mesenchymal cancers were acute leukaemias (FIG. 4A) and lymphomas (FIG. 4B). No sarcomas were seen in any of the Combi-tTA-Snail mice analysed, even though with ubiquitous expression of Combi-tTA-Snail. Detailed analysis of the epithelial tumour cells established the diagnosis as lung carcinomas (FIG. 5A), germ cell hyperplasias (FIG. 5B) and hepatocarcinomas (FIG. 5C). One type of carcinoma per animal was detected, although 20-25% of them also develop a hematopoietic neoplasia. The histological examination could not show dissemination of the carcinomas. However, histological analysis revealed marked leukaemic cell infiltration of hematopoietic and non-hematopoietic tissues. These leukaemic cells preferentially infiltrate kidney, liver, and lung, (FIG. 4C-E). Peripheral blood mononuclear cells from leukaemic mice were identified by flow cytometry using combination of specific antibodies. These studies defined the acute leukemias as acute myeloid leukaemias (FIG. 4A).

Transgenic non-human mammals of this type are thus useful, among other goals, for studying epithelial tumours, mesenchymal tumours and/or cancers, and DNA damage-based diseases as well as for evaluating potentially useful compounds for treating, diagnosing and/or preventing said pathologies. The animal is particularly useful as a model which faithfully reproduces disseminated human cancers.

The introduction of a DNA construct in which SNAIL is expressed in a way which allows regulation by an exogenous factor, into the genome of a model animal causes a genetic anomaly. In a particular embodiment, the genetic anomaly caused by the expression of the SNAIL transgene results in a tumour selected from an epithelial tumour and a mesenchymal tumour, or a DNA damage-based disease, or a disseminated cancer, in which case, the descendents are analysed to evaluate the existence of activated genes and/or genes created by the genetic anomaly associated with the pathology in question.

It appears from the inventors' results that SNAIL must be kept above a certain threshold level to achieve normal development. Consistent with this interpretation, Combi-tTA-Snail induced a tumorigenic but not migratory phenotype in MEFs. These findings indicate that SNAIL does not require tumour formation before dissemination can place. However, “increased” SNAIL expression induces cancer in mice with high frequency.

In particular, it has been found that the transgenic animals described herein develop disseminated cancers that replicate features of human cancer. Accordingly, these models are of immense utility in studying factors that affect, and preferably prevent, cancer dissemination. The expression “non-human mammal”, as used herein, includes any non-human animal belonging to the class of mammals. The non-human mammal is preferably a mouse but may be another mammalian species, for example another rodent, for instance a rat, hamster or a guinea pig, or another species such as a monkey, pig, rabbit, or a canine or feline, or an ungulate species such as ovine, caprine, equine, bovine, or a non-mammalian animal species. In a particular embodiment, the transgenic non-human animal provided by the invention is a murine animal. The term “murine” includes mice, rats, guinea pigs, hamsters and the like. In a preferred embodiment the murine animal is a rat or a mouse; most preferably the non-human mammal of the invention is a mouse.

Although the use of transgenic animals poses questions of an ethical nature, the benefit to man from studies of the types described herein is considered vastly to outweigh any suffering that might be imposed in the creation and testing of transgenic animals. As will be evident to those of skill in the art, drug therapies require animal testing before clinical trials can commence in humans and under current regulations and with currently available model systems, animal testing cannot be dispensed with. Any new drug must be tested on at least two different species of live mammal, one of which must be a large non-rodent. Experts consider that new classes of drugs now in development that act in very specific ways in the body may lead to more animals being used in future years, and to the use of more primates. For example, as science seeks to tackle the neurological diseases afflicting a ‘greying population’, it is considered that we will need a steady supply of monkeys on which to test the safety and effectiveness of the next-generation pills. Accordingly, the benefit to man from transgenic models such as those described herein is not in any limited to mice, or to rodents generally, but encompasses other mammals including primates. The specific way in which these novel drugs will work means that primates may be the only animals suitable for experimentation because their brain architecture is very similar to our own.

This aspect of the invention aims to reduce the extent of attrition in drug discovery and development. Whenever a drug fails at a late stage in testing, all of the animal experiments will in a sense have been wasted. Stopping drugs failing therefore saves test animals' lives. Therefore, although the present invention relates to transgenic animals, the use of such animals should reduce the number of animals that must be used in drug testing programmes and decrease attrition rates in clinical assays in humans.

The term “effector substance”, as used herein, refers to any substance which is capable of regulating the expression of the SNAIL gene when said substance is administered to the transgenic non-human mammal of the invention. These exogenously regulated expression systems are well known by a person skilled in the art (Maddison K., Clarke A R. 2005. New approaches for modelling cancer mechanisms in the mouse. J. Pathol. 205:181-193).

For the generation of the transgenic non-human mammal of the invention, a DNA construct containing the SNAIL gene (transgene) is made. Preferably, this gene will be introduced into the animal as a DNA construct, preferably comprising regulatory sequences. These regulatory sequences may be derived from humans, animals, prokaryotes or other species. In cases where the regulatory genes are not of human origin, the regulatory genes may be derived from the target animal, for example, the mouse. By regulatory genes is meant to include any promoter or enhancer sequences, 5′ or 3′ UTRs, poly-A termination sequences or other DNA sequences, that are necessary for transcription of the gene of interest. Transcripts used for insertion of human sequences are preferably terminated by a poly A motif. The invention may incorporate the endogenous promoter with the SNAIL coding gene so that the fidelity of wild type expression is retained, developmentally, temporally and in a tissue-specific manner. By “endogenous promoter” is meant the promoter that naturally directs expression of the gene of interest. The endogenous promoter may thus be the endogenous human promoter, or may alternatively be the promoter that is endogenous to that introduced gene in the transgenic animal subject. For example, in the case of transgenic mice, the expression of the human gene may be directed by the endogenous mouse promoter for that gene.

In a preferred embodiment of the invention, a non-human animal, such as a mouse, is made transgenic for SNAIL in a regulatable manner, and is also null for P53 expression (i.e. is P53−/−). An advantage of this aspect of the invention is that a cancer scenario can be recreated in the model organism, since most cancerous cells are P53−/−. Most surprisingly, the inventors have found that in this model, features of human cancer can be replicated in a non-human animal system. Although the inventors do not wish to be bound to any theory, it is thought likely that in normal non-cancerous cells, DNA damage leads to repression of SNAIL expression. However, P53 repairs the DNA damage such that cancer development does not occur. In the dual model of the invention, however, P53 is not present in the transgenic system and so cannot act to repair the DNA damage or act as a check on cancerous growth. Additionally, in this system, SNAIL expression is not repressed as would usually occur in the normal course of events, and as a result, disseminated cancers occur in the transgenic model in a manner that could not have been predicted.

It is reported herein that when mice deficient in p53 (also termed “p53 null mice” or “p53−/− mice”) were crossed with Combi-tTA-SNAIL mice, to yield Combi-tTA-SNAIL-p53−/− mice, it was found that these mice develop very large thymic lymphomas at an age of 2-3 months (FIG. 11). It was moreover found that these tumours infiltrated the lung, the heart, the mediastinal space and were essentially impossible to dissect. Micrographs of histological preparations of lymphoma, lung tumour and sebaceoma samples are shown in FIG. 12.

It was thus found that Combi-tTA-SNAIL-p53−/− mice reproduced the features of human cancers, also and in particular with respect to the dissemination and metastasis of malignant human cancers. These Combi-tTA-SNAIL-p53−/− mice thus represent an ideal model to develop therapies targeting dissemination controls. This model accurately replicates all of the features of disseminated cancer in the human and so is of utmost value to those seeking to find methods and compounds that prevent such dissemination occurring.

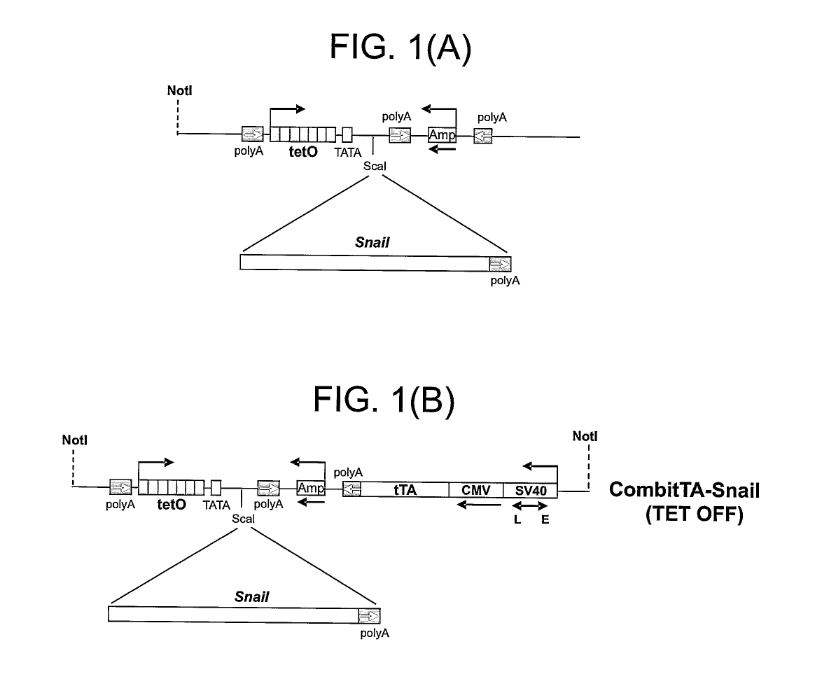

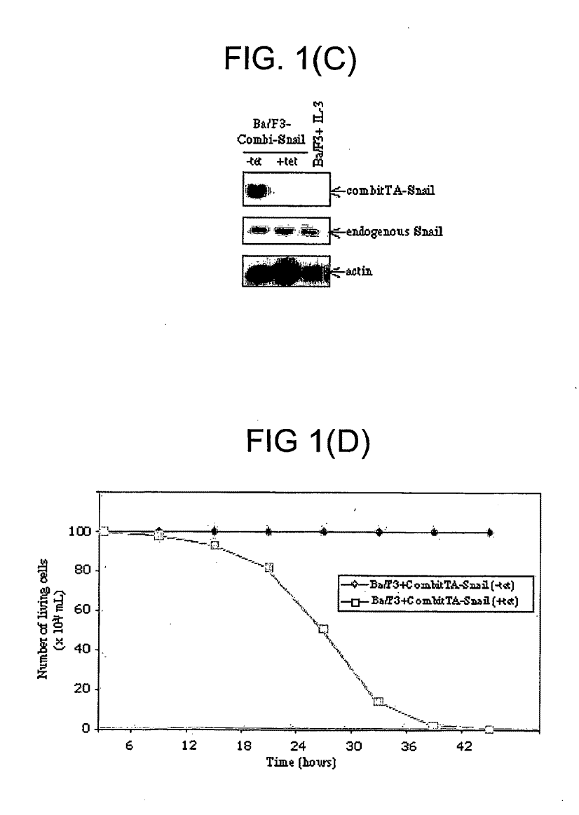

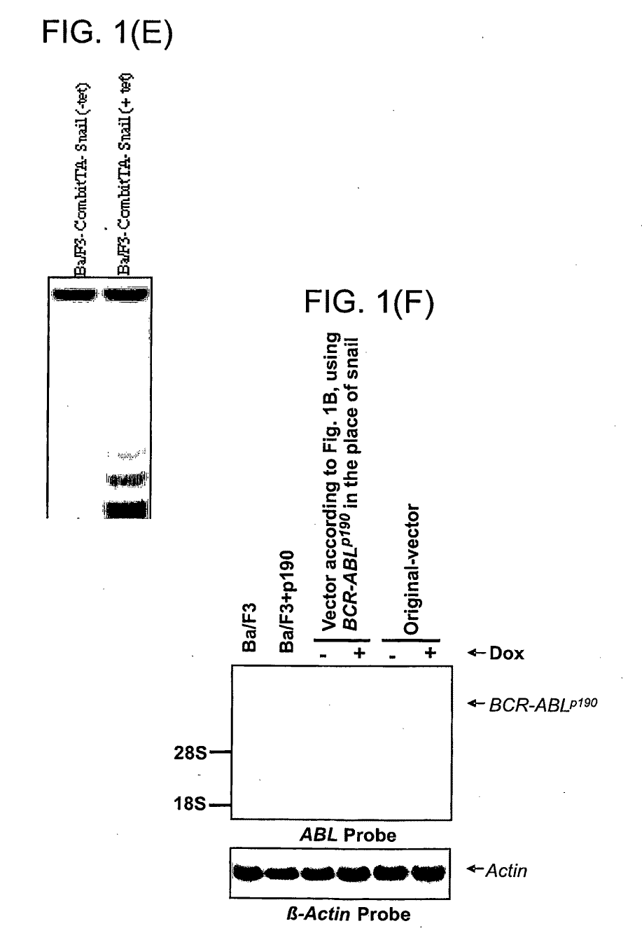

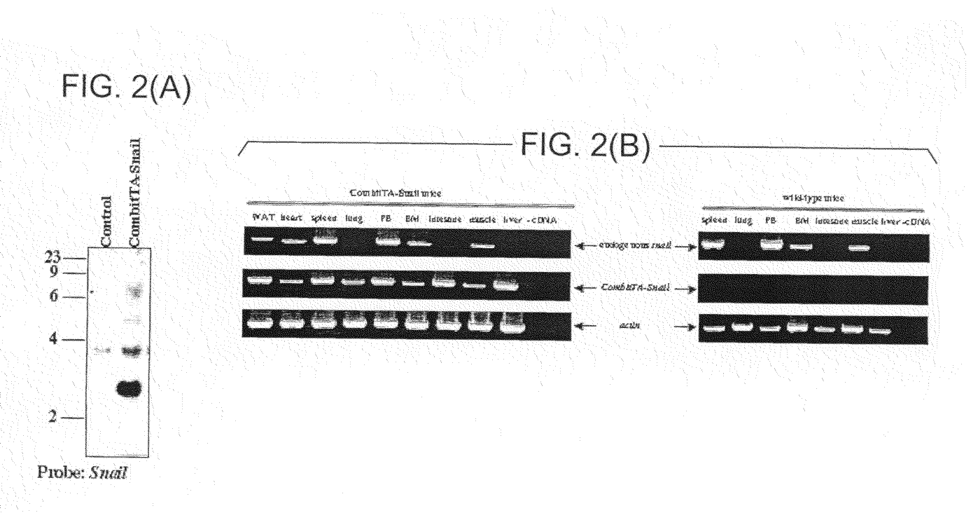

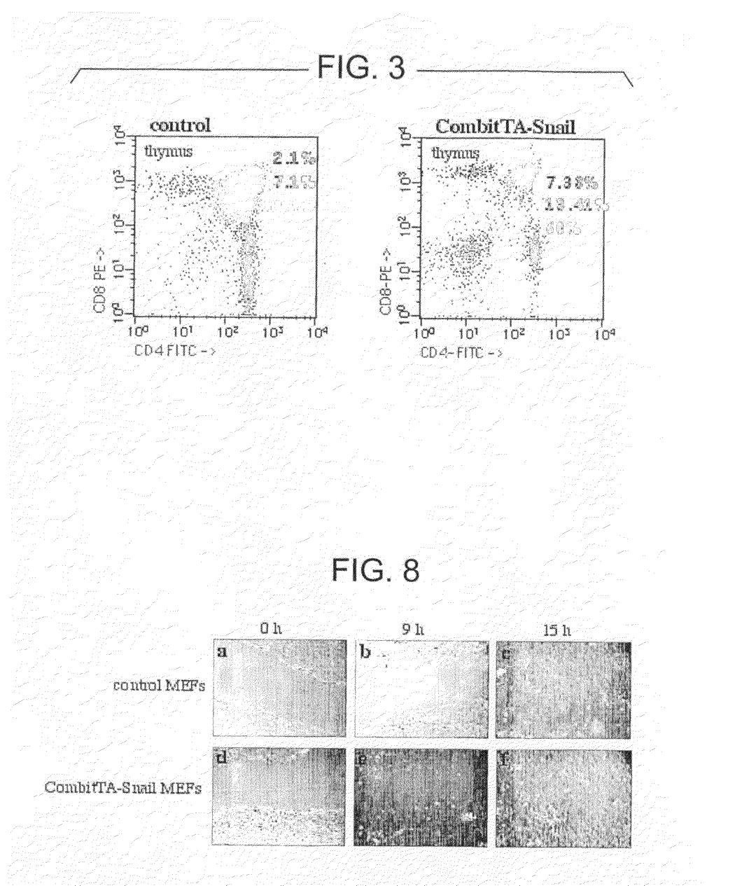

Preferably, said construct, hereinafter referred to as the DNA construct of the invention, thus comprises the SNAIL cDNA under the control of an expression system exogenously regulated by an effector substance. Suitable systems will be clear to those of skill in the art. In a preferred embodiment, the exogenously regulated expression system may be based on the tet-off system (Clontech), i.e. the Combi-tTA (Combi-tTA) vector system of Schultze et al. (Nature Biotechnology 14: 499-503, 1996), or a modified version thereof. A schematic representation of said DNA construct is shown in FIG. 1A. In this embodiment, the SNAIL gene is preferably under the control of the tet-operator (tetO) minimal promoter. In this case, the expression of the SNAIL transgene is exogenously regulated by tetracyclin or its derivatives such as doxycyclin, and the effector substance according to the invention is preferably tetracyclin or its derivatives such as doxycyclin. Thus, the SNAIL transgene of the trangenic non-human mammal according to the invention is preferably silenced in the presence of tetracyclin and activated in the absence of tetracyclin.

In one embodiment, the original Combi-tTA vector as described by Schultze et al. (Nature Biotechnology 14: 499-503, 1996) is preferably modified by the following steps: 1) removal of the tetO-luciferase cassette from said original vector, and 2) introduction of a cassette comprising the tetO minimal promoter and the SNAIL gene. Preferably said cassette should be introduced within the ampicillin resistance gene (referred to alternatively as Amp, bla, or the beta-lactamase gene) of the original vector disclosed in Schultze et al.

The inventors have improved upon the single-plasmid system of Schultze et al., (1996) containing the regulating and expression elements of the original binary tetracycline system to allow induction and tight control of gene expression by tetracycline in mice. The inventors found that the Schultze system requires some significant modification in order to allow a target gene to be efficiently expressed and appropriately silenced. For example, it has been found that without the modifications described herein, target gene expression is not silenced in the presence of tetracycline or, e.g., tetracyclin derivatives such as doxycyclin, probably because of read-through from the other promoters (e.g. CMV and SV40) that are present on the Schultze plasmid. Accordingly, one, preferably two or more polyA sequences are introduced in flanking positions around the target gene to ensure that this read-through problem is resolved.

Additionally, it has been found useful to introduce a TATA sequence in order to improve expression of the target protein from this system. Preferably, this TATA sequence lies between the tetO sequence and the target gene sequence.

Preferably, said introduced cassette comprises a poly-A sequence, the tetO promotor, a TATA box sequence, the target gene, further two poly-A sequences, an ampicillin resistance gene, and a fourth poly-A sequence. These elements are preferably arranged on said cassette in the aforementioned order. This modified construct, as described above, may be used for expression of any target gene in a manner which is regulated by tetracycline, or its derivatives such as doxycyclin, and forms an independent aspect of the present invention. Accordingly, this aspect of the invention provides a DNA construct adapted for the expression of a target gene in a way which allows regulation by an exogenous factor, said construct comprising an origin of replication, at least one promoter, at least one sequence capable of mediating regulation by an exogenous factor, at least one transactivator sequence and a sequence encoding the target gene, wherein the sequence encoding the target gene is flanked on both sides by at least one polyA sequence. Preferably, the flanking polyA sequences are situated so as to prevent read-through from the promoter sequences, such as in the configuration set out in FIG. 1. The construct may contain one, two or more flanking polyA sequences. It is not necessarily essential for the flanking polyA sequences to be directly contiguous with the sequence encoding the target gene. However, in a preferred embodiment, at least one polyA sequence is situated directly 5′ and directly 3′ to the sequence encoding the target gene.

By “polyA” sequence is meant a polyadenylation signal as known from eukaryotic genetics. Typically, polyadenylation sequences recognized by mammalian cells are regulatory regions located 3′ to the translation stop codon and thus, together with the promoter elements, flank the coding sequence. Examples of polyadenylation signals include those derived from SV40, although others will be known to those of skill in the art. Such sequences comprise runs of adenosine nucleotides, preferably between 10 and 500 nucleotides in length, more preferably between 50 and 200 nucleotides.

Preferably, the promoter comprises an SV40 promoter and/or a CMV promoter, more preferably both an SV40 promoter and a CMV promoter. Preferably the transactivator comprises the viral VP16 transactivator domain. More preferably transactivator comprises the viral VP16 transactivator domain fused to the tet-repressor protein. However, other transactivator systems will be known to those of skill in the art

Preferably, the construct additionally comprises a promoter sequence, preferably a TATA sequence, preferably situated upstream of the sequence encoding the target gene. Preferably, the promoter sequence lies between the sequence capable of mediating regulation by the exogenous factor and the sequence encoding the target gene.

Preferably, the sequence capable of mediating regulation by the exogenous factor is tetO, or a functional equivalent thereof, and the exogenous factor is tetracyclin, or a derivative thereof, such as doxycyclin.

Preferably, the sequence encoding the target gene is a gene implicated in predisposition to cancer, including oncogenes, and SNAIL. Other useful examples will be clear to those of skill in the art.

A preferred embodiment of this aspect of the invention is a construct based on that represented in FIG. 1 herein for SNAIL. In a particularly preferred embodiment, the target gene is SNAIL.

The cassette for insertion into the Schultze et al. Combi-tTA vector, preferably within the bla gene, in the construction of the transgenic non-human mammal according to the present invention, is shown in FIG. 1A. The final construct according to the present invention is the Combi-tTA vector resulting from the insertion of said cassette containing the SNAIL gene, and is referred to herein as the Combi-tTA-SNAIL vector. A preferred embodiment of said construct/vector is shown in FIG. 1B. Most preferably, the orientation of the poly-A sequence, the tetO promotor, the TATA box sequence, the SNAIL gene, the further poly-A sequences, the ampicillin resistance (beta-lactamase/bla) gene, and fourth poly-A sequence is as shown in FIGS. 1A and 1B, though, according to some embodiments, the Amp (bla) may also be in the opposite relative orientation.

The DNA construct of the invention is next introduced into a non-human mammal, or into a predecessor thereof, in an embryonic state, for example, in the state of a cell, or fertilized oocyte and, generally, not later than the G cell state.

There are different means conceived in the state of the art by which a sequence of nucleic acid can be introduced into an embryo of an animal such that it can be incorporated genetically in an active state, all of which can be applied for the generation of transgenic non-human mammals of the invention. A method consists of transfecting the embryo with said sequence of nucleic acid as occurs naturally, and selecting the transgenic animals in which said sequence has been integrated onto the chromosome at a locus that gives as a result the activation of said sequence. Another method implies modification of the nucleic acid sequence, or its control sequences, before introducing it into the embryo. Another method consists of transfecting the embryo using a vector that contains the nucleic acid sequence to be introduced.

In a particular embodiment, the introduction of the DNA construct of the invention in the germ line of a non-human mammal is performed by means of microinjection of a linear DNA fragment that comprises the activatable gene in fertilized oocytes of non-human mammals.

The fertilised oocytes can be isolated by conventional methods, for example, provoking the ovulation of the female, either in response to copulation with a male or by induction by treatment with the luteinising hormone. In general, a superovulation is induced in the females by hormonal action and they are crossed with males. After an appropriate period of time, the females are sacrificed to isolate the fertilised oocytes from their oviducts, which are kept in an appropriate culture medium. The fertilised oocytes can be recognised under the microscope by the presence of pronuclei. The microinjection of the linear DNA fragment is performed, advantageously, in the male pronucleus.

After the introduction of the linear DNA fragment that comprises the SNAIL construct of the invention in fertilised oocytes, they are incubated in vitro for an appropriate period of time or else they are reimplanted in pseudopregnant wet nursing mothers (obtained by making female copulate with sterile males). The implantation is performed by conventional methods, for example, anaesthetising the females and surgically inserting a sufficient number of embryos, for example, 10-20 embryos, in the oviducts of the pseudopregnant wet nursing mothers. Once gestation is over, some embryos will conclude the gestation and give rise to transgenic non-human mammals, which theoretically should carry the DNA construct of the invention integrated into their genome and present in all the cells of the organism. This progeny is the G0 generation and their individuals are the “transgenic founders”. The confirmation that an individual has incorporated the injected nuclear acid and is transgenic is obtained by analysing the individuals of the progeny. To do this, from a sample of animal material, for example, from a small sample from the animal's tail (in the event that it is, for example, a mouse) or a blood example, the DNA is extracted from each individual and analysed by conventional methods, for example, by PCR using the specific primers or by Southern blot or Northern blot analysis using, for example, a probe that is complementary to, at least, a part of the transgene, or else by Western blot analysis using an antibody to the protein coded by the transgene. Other methods for evaluating the presence of the transgene include, without limitation, appropriate biochemical assays, such as enzymatic and/or immunological assays, histological staining for particular markers, enzymatic activities, etc.

According to a preferred embodiment of the invention, the transgenic non-human mammal thus generated is preferably obtainable by the procedures mentioned above using the Combi-tTA-SNAIL vector. In this embodiment, the transgenic non-human mammal of the invention is referred to herein as a Combi-tTA-SNAIL mouse.

In general, in transgenic animals, the inserted transgene is transmitted as a Mendelian characteristic and so it is not difficult to establish the stable lines of each individual. If the G0 individuals are crossed with the parent strain (retrocrossing) and the transgene behaves with Mendelian characteristics, 50% of the progeny will be heterozygotic for the inserted transgene (hemizygotic). These individuals constitute the G1 progeny and a transgenic line that can be maintained indefinitely, crossing hemizygotics of the G1 generation with normal individuals. Alternatively, individuals of the G1 generation can be crossed among themselves to produce 25% homozygotics for the inserted transgene, 50% hemizygotics and 25% without the transgene provided the transgene does not affect the viability of the descendents.

The progeny of the transgenic non-human mammal of the invention, such as the progeny of a transgenic mouse provided by this invention can be obtained, therefore, by copulation of the transgenic animal with an appropriate individual, or by in vitro fertilization of eggs and/or sperm of the transgenic animals. As used in this description, the term “progeny” or “progeny of a transgenic non-human mammal” relates to all descendents of a previous generation of the transgenic non-human mammals originally transformed. The progeny can be analysed to detect the presence of the transgene by any of the aforementioned methods. The progeny of the transgenic non-human mammal of the invention, hereinafter referred to as the progeny of the transgenic non-human mammal of the invention, constitutes a further aspect of the present invention.

The invention also relates to a cell line of the transgenic non-human mammal of the invention or of the progeny of the transgenic non-human mammal of the invention, to a primary cell of the transgenic non-human mammal of the invention or of the progeny of the transgenic non-human mammal of the invention or to a tissue sample of the transgenic non-human mammal of the invention or of the progeny of the transgenic non-human mammal of the invention. Said cell line, primary cell or tissue sample, contains a DNA construct of the invention on its genome, i.e., a DNA construct containing the SNAIL gene. In a particular embodiment, said cell line, primary cell or tissue sample is a murine cell line, primary cell or tissue sample.

The transgenic non-human mammal of the invention, the progeny thereof, the cell line, primary cell and tissue sample provided by this invention, are useful for, among other applications, evaluating potentially useful compounds for treating and/or preventing a genetic anomaly, said genetic anomaly being associated with the development of epithelial or mesenchymal tumours and/or cancers or with DNA damage-based diseases.

Therefore, in other aspect, the invention refers to the use of the transgenic non-human mammal of the invention, or of the progeny thereof, for identifying potentially therapeutic compounds for the treatment of a tumour selected from an epithelial tumour and a mesenchymal tumour, and/or for the treatment of DNA damage-based diseases, or for evaluating the efficacy of therapy administered to a subject suffering from said tumour or DNA damage-based disease, or for monitoring the evolution of said tumour or DNA damage-based disease, or for affecting, preferably preventing, cancer dissemination.

### Drug Screening

The invention also refers to the use of the transgenic non-human mammal of the invention, its progeny, or of a cell line, a primary cell or a tissue sample from the transgenic non-human mammal of the invention or its progeny in the screening, identification, validation, optimization and/or evaluation of potentially useful compounds (candidate compounds) for the prevention treatment and/or diagnosis of a tumour selected from an epithelial tumour and a mesenchymal tumour, and/or for the treatment, prevention and/or diagnosis of a DNA damage-based disease, and/or for the treatment, prevention and/or diagnosis of cancer dissemination.

Therefore, in an aspect, the invention refers to a method for screening, searching, identifying, validating, optimizing, discovering, developing and/or evaluating compounds for the treatment and/or prevention of a mesenchymal or epithelial tumour or DNA damage-based disease or for repositioning known drugs or combinations of compounds, or for preventing dissemination, which comprises administering a candidate compound to a transgenic non-human mammal of the invention, or to its progeny, and monitoring the response.

The screening, searching, identifying, discovering, developing and/or evaluating of the candidate compound for the prevention and/or treatment of epithelial or mesenchymal tumours and/or cancers or a DNA damage-based disease, or preventing dissemination can be performed by administering the candidate compound to the transgenic non-human animal of the invention, at different doses, and evaluating the physiological response of the animal over time. The candidate compound can be administered to the transgenic non-human animal of the invention by any conventional and novel method, typically via oral or parenteral, depending, among other factors, on the chemical nature of the candidate compound. In some cases, it may be appropriate to administer the compound in question along with cofactors that enhance the effect of the compound.

In an embodiment, the above method comprises identifying, validating, optimizing and selecting a compound which inhibits or reduces the level of expression of SNAIL gene or its expression products (both transcription products and translation products, i.e., SNAIL mRNA or SNAIL protein). In order to achieve said aim, the candidate compound is administered to a transgenic non-human mammal of the invention or to its progeny, wherein the level of SNAIL expression products in a tissue sample is known, and, subsequently, the level of SNAIL expression products in said tissue is quantified, and a compound which is able to inhibit or reduce the level of SNAIL expression product is selected.

The quantification of the SNAIL expression products is carried out in a manner similar to that indicated in the method for determining the invasive and/or metastatic capacity of an epithelial or mesenchymal tumour.

In another aspect, the invention refers to a method for screening, searching, identifying, validating, optimizing, discovering, developing and/or evaluating compounds for the treatment and/or prevention of a mesenchymal or epithelial tumour or DNA damage-based disease or for repositioning known drugs or combinations of compounds, or for preventing cancer dissemination, which comprises contacting a cell line, or a primary cell, or a tissue sample of the transgenic non-human mammal of the invention, or its progeny, and monitoring the response.

The screening, searching, identifying, validating, optimizing, discovering, developing and/or evaluating of the candidate compound for the prevention and/or treatment of epithelial or mesenchymal tumours and/or cancers or a DNA damage-based disease or for preventing dissemination can be performed by adding the candidate compound to a culture medium containing cells from a cell line or from primary cells or from tissue samples provided by the present invention, for an appropriate period of time, at different concentrations, and evaluating the cellular response to the candidate compound over time using appropriate biochemical and/or histological assays. In an alternative embodiment, cells may be used that are transfected with a construct that expresses SNAIL in a mariner regulated by an exogenous substance. In a preferred embodiment, the Combi-TA SNAIL vector is used, as described herein. Examples of suitable cells include MEFs. At times, it may be necessary to add the compound in question to the cellular culture medium along with cofactors that enhance the effect of the compound.

In an embodiment, the above method comprises identifying and selecting a compound which inhibits or reduces the level of expression of SNAIL gene or its expression products (both transcription products and translation products, i.e., SNAIL mRNA or SNAIL protein). In order to achieve said aim, the candidate compound is contacted with a cell line, or with a primary cell, or with a tissue sample of the transgenic non-human mammal of the invention, or its progeny, wherein the level of SNAIL expression products in said cell line, primary line or tissue sample is known, and, subsequently, the level of SNAIL expression products in said tissue is quantified, and a compound which is able to inhibit or reduce the level of SNAIL expression products is selected.

The quantification of the SNAIL expression products is carried out in a manner similar to that indicated in the method for determining the invasive and/or metastatic capacity of an epithelial or mesenchymal tumour.

When a compound inhibits or decreases the levels of the SNAIL expression products or reverts the effects of the increased expression of said gene or the activity of SNAIL protein, this compound becomes a candidate for cancer therapy, especially for treating and/or preventing epithelial or mesenchymal tumours and/or cancers, or a candidate for treating and/or preventing DNA damage-based disease.

Illustrative, non limitative, examples of compounds which inhibit or decrease the levels of the SNAIL mRNA include antisense SNAIL mRNA, ribozymes, triple helix molecules, small interference RNA (siRNA), etc.

Illustrative, non limitative, examples of compounds which inhibit or decrease the levels of the SNAIL protein include antibodies anti-SNAIL, enzymes or proteins which regulate the activity of SNAIL protein, etc.

In other aspect, the invention refers to the use of a compound which inhibits or decreases the levels of the SNAIL expression products or reverts the effects of an increased level of SNAIL expression products in the manufacture of a pharmaceutical composition for prevention and/or treatment of a tumour selected from an epithelial tumour or a mesenchymal tumour, or of a DNA damage-based disease. Illustrative, non limitative, examples of said compounds include antisense SNAIL mRNA, ribozymes, triple helix molecules, small interference RNA (siRNA), antibodies anti-SNAIL, enzymes or proteins which regulate the activity of SNAIL protein, etc.

### Pharmaceutical Compositions

Further, in other aspect, the invention refers to a pharmaceutical composition comprising a therapeutically effective amount of a compound which inhibits or decreases the levels of the SNAIL expression products or reverts the effects of an increased level of SNAIL expression products together with one or more pharmaceutically acceptable excipients and/or carriers. The excipients, carriers and auxiliary substances must be pharmaceutically and pharmacologically tolerable, so that they can be combined with other components of the formulation or preparation and do not cause adverse effects in the treated organism. The pharmaceutical compositions or formulations include those which are suitable for oral or parenteral (including subcutaneous, intradermal, intramuscular or intravenous) administration, although the best administration route depends on the condition of the patient and the nature of the compound to be administered. The formulations can be in the form of single doses. The formulations are prepared according to methods known in the pharmacology field. The active substance amounts to administer may vary according to the particularities of the therapy. The pharmaceutical composition of the invention can also comprise one or more active ingredients useful for treating cancer or DNA damage-based diseases, such cytotoxic agents, etc.

In an embodiment, the pharmaceutical composition of the invention comprises a vector comprising a therapeutic compound suitable for the treatment and/or prevention of a mesenchymal or epithelial tumour or DNA damage-based disease. Said vector can be a viral vector or a non-viral vector. Illustrative, non limitative, examples of said therapeutic compound include antisense SNAIL mRNA, ribozymes, triple helix molecules, small interference RNA (siRNA), antibodies anti-SNAIL, enzymes or proteins which regulate the activity of SNAIL protein, etc.

It is important to mention that the development of molecular and pharmacological therapeutics to successfully treat and prevent pathologies such as cancer, will allow a precise assessment of the therapeutic potential of any strategy before the application in human therapy.

### Kits

In other aspect, the invention refers to a kit for carrying out the present invention. Thus, in an embodiment, the kit of the present invention comprises an antibody that specifically recognizes SNAIL protein in a suitable packing. In another embodiment the kit of the invention comprises a primer pair designed to specifically amplify a nucleic acid having a sequence that is specific to the SNAIL. The sequence of the primer pair can be determined from the sequence of the corresponding SNAIL gene by employing bioinformatic tools. These kits can be employed to determine the invasive and/or metastatic capacity of an epithelial or mesenchymal tumour, or the local growth capacity of an epithelial or mesenchymal tumour, or to in vitro diagnose the presence of a condition in a subject, said condition being selected from an epithelial tumour, a mesenchymal tumour and a DNA damage-based disease, or to determine the stage or severity of said condition in a subject, or to determine the predisposition of a subject to develop said condition, or to monitor the effect of the therapy administered to a subject with said condition, or for screening, searching, identifying, discovering, developing and/or evaluating compounds for the treatment, prevention and/or diagnosis of a mesenchymal or epithelial tumour or DNA damage-based disease.

The following examples illustrate the invention and should not be considered limiting the scope thereof.

### Example

### Cancer Development Induced by Graded Expression of SNAIL in Mice

### I. Materials and Methods

Generation of transgenic mice and treatments. The cDNA for mouse SNAIL was cloned into Combi-tTA vector (Schultze, N., Burki, Y., Lang, Y., Certa, U., & Bluethmann, H. Efficient control of gene expression by single step integration of the tetracycline system in transgenic mice. Nature Biotechnology 14: 499-503 (1996)). As it was found that the original Combi-tTA vector as published by Schultze et al. in fact did not allow efficient regulation of the tet operator both in vivo and in vitro, the following modifications were introduced: 1) the tetO-luciferase cassette was removed from said original Combi-tTA vector, and 2) a cassette comprising the a poly-A sequence, the tetO minimal promotor, a TATA box sequence, the SNAIL gene, further two poly-A sequences, an ampicillin resistance gene, and a fourth poly-A sequence was introduced within the ampicillin resistance gene (referred to alternatively as Amp, bla, or the beta-lactamase gene) of the original vector disclosed in Schultze et al.
Linear DNA fragments for microinjection were obtained by NotI digestion and injected into CBAxC57BL/6J fertilized eggs (Manipulating the mouse embryo, a laboratory manual. Second Edition. Hogan, Beddington, Costentine, Lacy. CSHL PRESS, 1994). Transgenic mice were identified by Southern analysis of tail snip DNA after EcoRI digestion as described previously (García-Hernandez et al., 1997. Murine hematopoietic reconstitution after tagging and selection of retrovirally transduced bone marrow cells. Proc. Natl. Acad. Sci. USA 94, 13239-13244). Detection of the transgene was performed using the mouse SNAIL cDNA. Founder mice were crossed to C57BL6 mice for five generations to establish co-isogenic transgenic mice. Similar phenotypic features were seen in all assays for both of the Combi-tTA-Snail transgenic lines generated. Mice aged 5-6 weeks were irradiated using a cesium source and maintained in microisolator cages on sterilized food and acidified sterile water.
Histological analysis. Mice included in this study were subjected to standard necropsy. All major organs were examined under the dissecting microscope, and samples of each organ were processed into paraffin, sectioned and examined histologically. All tissue samples were taken from homogenous and viable portions of the resected sample by the pathologist and fixed within 2-5 minutes. For comparative studies, age-matched mice were used (wild-type or Combi-Snail mice with continuous presence of tetracycline). Cell culture. Cell lines used include Ba/F3 cells (Palacios and Steinmetz, 1985. IL-3 dependent mouse clones that express B-220 surface antigen, contain Ig genes in germ-line configuration, and generate B-lymphocytes in vivo. Cell 41:727). Cells were maintained in Dulbecco's modified Eagle's medium (DMEM) (Boehringer Ingelheim) supplemented with 10% foetal calf serum (FCS). When required, 10% WEHI-3B-conditioned medium was added as a source of IL-3.
Cell transfection and Cell Survival Assay. Ba/F3 cells were transfected by electroporation (960 μF, 220 V) with 20 μg of each Combi-tTA-Snail. The neomycin-resistant pool of cells (Ba/F3+Combi-tTA-Snail) were analysed by RT-PCR for Combi-tTA-Snail expression in the presence and in the absence of tetracycline (20 ng/ml). These cells were resistant to IL-3 withdrawal when grown in the absence of tetracycline. Cells were screened for resistance to IL-3 withdrawal and cell viability was determined by trypan blue exclusion.
Culture of MEFs. Heterozygous p53+/− (Jackson Laboratories) and p21+/− (provided by M. Serrano) mice (Martin-Caballero et al. 2004. Oncogene, 23: 8231-8237) were crossed to obtain wild-type (wt) and null p53−/− and p21−/− embryos, respectively. Primary embryonic fibroblasts were harvested from 13.5 d.p.c. (days postcoitum) embryos. Head and organs of day 13.5 embryos were dissected; fetal tissue was rinsed in phosphate-buffered saline (PBS), minced, and rinsed twice in PBS. Foetal tissue was treated with trypsin/EDTA (ethylendiaminetetraacetic acid) and incubated for 30 min at 37° C. and subsequently dissociated in medium. After removal of large tissue clumps, the remaining cells were plated out in a 175 cm² flask. After 48 hours, confluent cultures were frozen down. These cells were considered as being passage 1 MEFs (mouse embryonic fibroblasts). For continuous culturing, MEF cultures were split 1:3. MEFs were grown at 37° C. in Dulbecco's-modified Eagle's medium (DMEM; Boehringer Ingelheim) supplemented with 10% heat-inactivated FCS (Boehringer Ingelheim). All the cells were negative for mycoplasma (MycoAlert™ Mycoplasma Detection Kit, Cambrex).
DNA-damage experiments. Cells were plated at 10⁶ cells per 10-cm dish, and the day after, they were treated with 0.2 μg/mL of doxorubicin (Sigma). After 12 hours, cells were collected for RNA preparation.
Low molecular weight DNA analysis. Low molecular weight DNA was isolated as follows. Cells were collected into 1.5 ml of culture medium and microcentrifuged for 1 minute at 1,500 rpm (400×g), and the pellet was suspended in 300 μl of proteinase K buffer. After overnight incubation at 55° C., DNA was ethanol-precipitated, suspended in 200 μl of TE buffer (Tris-EDTA) pH 7.4 containing 50 μg/ml of RNase A, and incubated at 37° C. for 2 hours. DNA was extracted with phenol and chloroform and precipitated with ethanol. Aliquots of DNA (2 μg) were end-labelled with a32-dCTP and electrophoresed on 2% agarose gels. After electrophoresis, the gel was blotted onto Hybond-N (Amersham) and autoradiographied for 2 hours at −70° C.
Reverse Transcription-PCR (RT-PCR) and Real-time PCR quantification. To analyze expression of Combi-tTA-Snail and endogenous SNAIL in mouse cell lines and mice, RT-PCR was performed according to the manufacturer's protocol in a 20-μl reaction containing 50 ng of random hexamers, 3 μg of total RNA, and 200 units of Superscript II RNase H⁻ reverse transcriptase (GIBCO/BRL). The sequences of the specific primers were as follows:

| | |
|---|---|
| Combi-polyA-B1: | 5′-TTGAGTGCATTCTAGTTGTG-3′; |
| mSnailF: | 5′-CAGCTGGCCAGGCTCTCGGT-3′; |
| mSnailB: | 5′-GCGAGGGCCTCCGGAGCA-3′. |

Amplification of β-actin RNA served as a control to assess the quality of each RNA sample. The PCR conditions used to amplify Combi-tTA-Snail and endogenous SNAIL were as follows: 94° C. for 1 minute, 56° C. for 1 minute, and 72° C. for 2 minutes for 40 cycles for Combi-tTA-Snail and 30 cycles for endogenous SNAIL, respectively. The PCR products were confirmed by hybridization with specific internal probes. Real-time quantitative PCR was carried out for the quantification of both Combi-tTA-Snail and endogenous SNAIL. Fluorogenic PCRs were set up in a reaction volume of 50 μl using the TaqMan PCR Core Reagent kit (PE Biosystems). cDNA amplifications were carried out in a 96-well reaction plate format in a PE Applied Biosystems 5700 Sequence Detector. Thermal cycling was initiated with a first denaturation step of 10 minutes at 95° C. The subsequent thermal profile was 40 cycles of 95° C. for 15 s, 56° C. for 30 s, 72° C. for 1 minute. Multiple negative water blanks were tested and a calibration curve determined in parallel with each analysis. The β-actin endogenous control (PE Biosystem) was included to relate both Combi-tTA-Snail and endogenous SNAIL to total cDNA in each sample.

Phenotype analysis. The following anti-mouse monoclonal antibodies from Pharmingen were used for cytometry staining: CD45R/B220, IgM, Mac1, Gr-1, CD4, and CD8. Single cell suspensions from the different tissue samples obtained by routine techniques were incubated with purified anti-mouse CD32/CD16 (Pharmingen) to block binding via Fc receptors and with an appropriate dilution of the different antibodies at room temperature or 4° C., respectively. The samples were washed twice with PBS and resuspended in PBS. Dead cells in samples were excluded by propidium iodide staining. The samples and the data were analysed in a FACScan using CellQuest software (Becton Dickinson).

Tumorigenicity assay. To test the tumorigenicity of the various Combi-tTA-Snail cancers and MEFs, 4- to 6-week-old athymic (nude) male mice were injected subcutaneously on both flanks with 10⁶ cells resuspended in 200 μl of PBS. The animals were examined for tumour formation every week.
Luciferase assays. The approximately 4935-bp upstream promoter sequence of SNAIL was isolated from a P1 clone containing the SNAIL gene (Genome Systems) and cloned into the luciferase reporter plasmid pGL3-basic (Promega) and termed PSNAIL-4935. Human MYB cDNA was generated by RT-PCR and nucleotide sequence was verified by sequencing and the cDNA was cloned into the expression plasmid pEF-BOS (Mizhusima, S. and Nagata, S. 1990. Nucleic acids Research, 18: 5322) For reporter assays, U2OS cells (Yao, F. and Schaffer, P A. 1995. J. Virol, 69: 6249-6258) were transfected using Dual-Luciferase (Promega) with normalization to Renilla luciferase, and mean±standard error was determined from at least three data points. U2OS cells were maintained in Dulbecco's Modified Eagle Medium supplemented with 10% FCS.
Northern blot analysis. Total cytoplasmic RNA of different MEFs and spleen tissues from both untreated and 5 Gy-irradiated wild-type and the p53−/− mice was glyoxylated and fractionated in 1.4% agarose gels in 10 mM Na₂HPO₄ buffer (pH 7.0). After electrophoresis, the gel was blotted onto Hybond-N (Amersham), UV-cross-linked, and hybridised to ³²P-labelled mouse Snail cDNA probe. Loading was monitored by reprobing the filter with an ARPP-P0 probe (Sage, J. et al., 2000. Gens Dev. 14: 3037-3050).
Western blot analysis. Bone marrow (BM) cells were collected by flushing the marrow cavity of femurs. Western blot assays were done using extracts from 1×10⁷ BM cells per lane. Extracts were normalized for protein content by Bradford analysis (Bio-Rad Laboratories, Inc., Melville, N.Y., USA) and Coommasie blue gel staining. Lysates were run on a 10% SDS-PAGE gel and transferred to a PVDF membrane (Polyvinylidene Difluoride). After blocking, the membrane was probed with the following primary antibodies: Mouse p53 was detected using the antibody FL-393 (Santa Cruz), and the polyclonal antibody C-11 (Santa Cruz) was used to detect actin. Reactive bands were detected with an ECL system (Amersham).
Migration assays. The migratory/motility behaviour of transfectant cells was analyzed by the wound assay. Monolayers of confluent cultures were lightly scratched with a Gilson pipette tip and, after washing to remove detached cells, the cultures were observed at timely intervals as previously described (Cano et al., 2000. The transcription factor Snail controls epithelial-mesenchymal transitions by repressing E-cadherin expression. Nature Cell Biology 2: 76-83).

### II. Results

### Derivation of Combi-tTA-Snail Mice

In order to determine the effect of upregulation of SNAIL expression in cancer development, transgenic mice, using the Combi-tTA system, in which the expression of SNAIL gene could be exogenously regulated, were generated. This system, which has the transactivator and the tet-operator minimal promoter driving the expression gene unit on a single plasmid, ensures the integration of the transactivator and reporter gene units in equal copy numbers in a direct cis-configuration at the same chromosomal locus and prevents genetic segregation of the control elements during breeding.

However, initial experiments using the Combi-tTA vector as originally described by Schultze et al. indicated that tight regulation of the transgene was in fact not possible using this original vector. This became evident in initial experiments with said original vector using BCR-ABL^{p190} as a transgene. BCR-ABL^{p190} is normally downregulated in the presence of doxycyclin (a tetracyclin derivative). However, using the original vector as described by Schultze et al, expression of BCR-ABL^{p190} was still observed in the presence of doxycyclin, indicating leaky expression of the transgene even under conditions that should not allow expression. This effect is shown in the two lanes at the far right of the Northern Blot of FIG. 1F, labelled “original vector”. Regulation of the transgene was only possible when the additional modifications described for the Combi-tTA-SNAIL construct under Materials and Methods and in FIGS. 1A and 1B were introduced. In brief, it was found that a cassette comprising additional features alongside the desired transgene (e.g. the SNAIL or BCR-ABL^{p190} gene, or any other desired “genetic alteration”) was introduced within the bla gene of the original vector, to replace the tetO-luciferase cassette of the original vector, tight regulation of the transgene both in vivo and in vitro became possible. Thus it was found that it was necessary to replace the tetO-luciferase cassette of the original vector by a cassette comprising a poly-A sequence, the tetO promotor, a TATA box sequence, the desired transgene (e.g. the SNAIL or BCR-ABL^{p190} gene, or any other desired “genetic alteration”), further two poly-A sequences, an ampicillin resistance gene, and a fourth poly-A sequence, in the order and orientation shown for the Combi-tTA-SNAIL construct of the present invention in FIGS. 1A and 1B. FIG. 1F shows that tight regulation of the transgene became possible in a cellular model when said cassette was used to replace the tetO-luciferase cassette of the original vector, in the example of the BCR-ABL^{p190} as the transgene. Therefore, in the construction of the Combi-tTA-SNAIL vector, the mSNAIL gene was inserted into the Combi-tTA vector under the control of the tetO-minimal promoter alongside the aforementioned additional features, as shown in FIGS. 1A and B., Combi-tTA.

Regulation of the expression of the SNAIL gene of the Combi-tTA-SNAIL vector was analysed in a cell system, using a murine hematopoietic precursor Ba/F3 cell line. In the absence of tetracycline, the tet-repressor protein (fused to the viral VP16 transactivator domain) binds to an engineered tet-operator minimal promoter and activates SNAIL transcription (Combi-tTA-Snail). In the presence of tetracycline, binding is abolished and the promoter silenced (FIG. 1A). Combi-tTA-Snail expression was determined in transfected Ba/F3 cells after culturing for two days in the presence or absence of tetracycline (FIG. 1B). Combi-tTA-Snail was detected in Ba/F3 cells without tetracycline but not in cells cultured with tetracycline (20 ng/ml). In vitro studies have previously shown that Snail confers resistance to cell death induced by the withdrawal of survival factors. The physiological relevance of the Combi-tTA-Snail suppression was confirmed in vitro by assaying survival of Ba/F3 cells expressing Combi-tTA-Snail 24 hours after IL-3 withdrawal. The effects of SNAIL expression on cell growth were evaluated by analyzing internucleosomal DNA cleavage leading to the formation of DNA ladders in agarose gels, which is a hallmark of apoptosis. Normally, SNAIL expression protects Ba/F3 cells from apoptosis following IL-3 withdrawal (FIGS. 1C-D) and the level of Combi-tTA-Snail expression was sufficient in Ba/F3 cells to prevent cell death. The sensitivity to IL-3 removal was restored by the addition of tetracycline (FIGS. 1C-D).

Three founder transgenic lines for Combi-tTA-mSnail (59A, 59B, and 59C) (FIG. 2A) were generated and two founder lines, 59A and 59B, showed germline transmission of the transgene (Table I). In both lines, the Combi-tTA-Snail expression was detected in all tissues analyzed (FIG. 2B). The Combi-tTA-Snail expression was the result of transactivation as the suppression of expression to undetectable values was confirmed when mice were supplied with tetracycline in their drinking water (See below, FIG. 6A).

TABLE I
Incidence and age of tumour-onset in Combi-tTA-Snail mice

| Transgenic | Mice | Mice with | Age in months at | |
|---|---|---|---|---|
| line | autopsied^{a} | tumour (%)^{b} | tumour onset | Tumour type (%) |
| IS59A | 34 | 34 (100) | 7-11 | AML (40%) |
| | | | | Lymphoma (50%) |
| | | | | Lung carcinoma (12%) |
| | | | | Hepatocarcinoma (10%) |
| | | | | Germ cell hyperplasia (15%) |
| IS59B | 29 | 29 (100) | 5-10 | AML (35%) |
| | | | | Lymphoma (40%) |
| | | | | Lung carcinoma (15%) |
| | | | | Hepatocarcinoma (15%) |
| | | | | Germ cell hyperplasia (15%) |
| IS59C^{c} | 1 | 1 (NA) | 1 | Leukaemia |

^{a}Number of mice during or after the period of cancer.
^{b}Number of mice killed with cancer and percentage of tumour incidence.
^{c}No lineage established

### Combi-tTA-Snail Mice Show No Morphological Abnormalities

Cohorts of Combi-tTA-Snail mice were generated to analyze the effect of the SNAIL expression in vivo. A total of 63 transgenic animals (34 mice corresponded to line 59A and 29 mice to line 59B) were analyzed in detail and similar phenotypic features were seen in both lines. Combi-tTA-Snail mice were born alive without overt morphological abnormalities, and were fully fertile with no differences apparent in the progeny. Autopsy of pups, including extensive histological analysis, revealed no abnormality of the kidneys, skin, liver, brain, lung or gastrointestinal tract of Combi-tTA-Snail mice, indicating that this level of overexpression of SNAIL does not perturb normal embryonic development.

### Cancer Development in Combi-tTA-Snail Mice

Inventors further analyzed whether the Combi-tTA-Snail mice develop cancer. All Combi-tTA-Snail mice became unwell from approximately 5-7 months of age onward (Table I) with clinical manifestations that included decreased physical activity, tachypnea, pilo-erection, shivering, and sustained weight loss, prior to sacrifice. The cancers were from both mesenchymal and epithelial origin (Table I). The mesenchymal cancers were acute leukaemias (FIG. 4A) and lymphomas (FIG. 4B). No sarcomas were seen in any of the Combi-tTA-Snail mice analysed, even though with ubiquitous expression of Combi-tTA-Snail. Detailed analysis of the epithelial tumour cells established the diagnosis as lung carcinomas (FIG. 5A), germ cell hyperplasias (FIG. 5B) and hepatocarcinomas (FIG. 5C). One type of carcinoma per animal was detected, although 20-25% of them also develop a hematopoietic neoplasia. The histological examination could not show dissemination of the carcinomas. However, histological analysis revealed marked leukaemic cell infiltration of hematopoietic and non-hematopoietic tissues. These leukaemic cells preferentially infiltrate kidney, liver, and lung, (FIG. 4C-E). Peripheral blood mononuclear cells from leukaemic mice were identified by flow citometry using combination of specific antibodies. These studies defined the acute leukemias as acute myeloid leukaemias (FIG. 4A).

To test the malignant potential of cells from the Combi-tTA-Snail mice, 1×10⁶ peripheral blood blast cells from Combi-tTA-Snail leukaemias were injected subcutaneously into twelve 40-day old nude mice. All twelve mice developed progressive tumours within 4-7 weeks of transplantation. The tumours in the nude mice were histologically-identical to the original leukaemias. Overall, these data indicate that Snail is able to induce cancer development.

In Vivo Suppression of Snail does not Block Cancer Development

The above results support the view that SNAIL expression is enough to induce cancer development. Therefore abolition of SNAIL overexpression might be expected to either halt or reduce the growth and/or spread of the SNAIL-expressing cells. To assess this, forty leukaemic Combi-tTA-Snail mice were evaluated for disease progression by flow cytometry prior to and following administration of tetracycline (4 g/L in the drinking water for 2 weeks, a dose sufficient to suppress of exogenous SNAIL expression) (FIG. 6A). None of the Combi-tTA-Snail mice exhibited amelioration of the leukaemic phenotype despite complete CombiTA-Snail suppression: Flow cytometry analysis identified the persistence of leukaemic cells in the peripheral blood (FIG. 6B) with infiltration of non-hematopoietic tissues evident on histology (FIG. 6C). Autopsy of these animals identified carcinomas (FIG. 6C). Thus, these results show that the alterations induced by SNAIL are irreversible.

A Limited Amount of Snail mRNA was Expressed in Combi-tTA-Snail MEFs and Mice.

In order to analyse the molecular basis underlying cancer development in Combi-tTA-Snail mice, the expression of transgene-encoded SNAIL in the spleen and in primary mouse embryonic fibroblasts (MEFs) derived from Combi-tTA-Snail embryos, where the endogenous SNAIL is expressed, was examined (FIG. 7A). The expression level of transgene-encoded SNAIL in spleen and MEFs of mice with respect to the endogenous expression was increased to 20% of wild-type levels (FIG. 7A). A limited amount of SNAIL was expressed in all tissues examined. In fact, the expression of transgene-encoded SNAIL was present in epithelium of Combi-tTA-Snail mice (FIG. 2B) and in the carcinomas appearing in Combi-tTA-Snail mice (FIG. 7B). Thus, these mice are an ideal in vivo model to study the consequences of low levels of Snail. In conclusion, our genetic studies point, for the first time, to the critical role for an appropriate expression level of an essential EMT regulator in cancer mouse development.

### Combi-tTA-Snail Induces a Tumorigenic but not Migratory Phenotype in MEFs

The above results suggest that Combi-tTA-Snail is not present at a level sufficient to alter EMT in Combi-tTA-Snail mice. To study the migratory properties of Combi-tTA-Snail MEFs, a wound culture assay was analysed, where Combi-tTA-Snail MEFs showed a similar migratory behaviour to control MEFS. Approximately 80% of the wound surface was colonized by both control and Combi-tTA-Snail MEFs 15 hours after the wound was made (FIG. 8A). To test the tumorigenic properties of the Combi-tTA-Snail MEFs, 1×10⁶ control and Combi-tTA-Snail cells were injected subcutaneously into 40-day old nude mice. Mice injected with control MEFs did not develop tumours (0 out of 10). However, Combi-tTA-Snail MEFs gave rise to tumours within 5-9 weeks of transplantation at the injection sites (10 out of 10). These results indicate that low levels of the transcription factor Snail induce a tumorigenic but not migratory phenotype in MEFs. In fact, metastasis was observed in Combi-tTA-Snail mice with carcinomas. Thus the transgene-encoded. SNAIL may not be present at a level sufficient to alter EMT in Combi-Snail mice, what could explain why Combi-tTA-Snail mice show no morphological abnormalities, but this level of expression was enough to produce cancer.

### Radioprotective Potential of Combi-tTA-Snail Mice in Response to γ-Irradiation

In order to investigate the in vivo radioprotective potential of Combi-tTA-Snail in response to DNA damage induced by γ-irradiation, Combi-tTA-Snail and control mice were irradiated at 950 rads (1 rad=0.01 Gy). As shown in FIG. 9A, Combi-tTA-Snail mice survive longer than control mice. These results indicate that Combi-tTA-Snail expression results in increased radioprotection.

It is known that exposure to ionizing radiation causes an increase in the intracellular levels of p53, and in vitro studies have also shown that aberrant overexpression of SNAIL and SLUG alters the response to genotoxic stress by increasing the level of p53. In order to investigate whether the radioprotective potential of Combi-tTA-Snail was based on interference with p53 activation, p53 protein levels at different time points in bone marrow cells derived from both Combi-tTA-Snail and control mice after DNA damage induced by γ-irradiation were measured (FIG. 9B). The activation of p53 in both control and Combi-tTA-Snail cells was similar (FIG. 9B), indicating that p53 regulation in response to DNA damage is not affected in Combi-tTA-Snail cells.

DNA Damage Regulates SNAIL mRNA Expression

The above results suggested that SNAIL expression protects cells from DNA damage. This led inventors to investigate whether DNA damage regulates SNAIL expression. MEFs were used as a model for in vitro studies to determine whether Snail has a functional role in response to DNA damage-mediated cellular activities (FIG. 10A). MEFs of different genotypes were treated with the chemotherapeutic agent, doxorubicin, with causes DNA damage. The expression of the p53 target gene p21 was used as a positive control. As shown in FIG. 10A, DNA damage inhibits expression of SNAIL in MEFs in a p53-independent manner. To confirm this result, approximately 4,935 base-pairs of the promoter region of the human SNAIL gene were cloned upstream of a luciferase reporter gene (pGL3-basic). To directly assess the ability of p53 to activate transcription from DNA sequences present in the SNAIL promoter, an expression vector containing a human p53 cDNA (Norris P S, Haas M. A fluorescent p53GFP fusion protein facilitates its detection in mammalian cells while retaining the properties of wild-type p53. Oncogene. 1997; 15(18):2241-2247) was co-transfected into U2OS cells along with the reporter vector containing the SNAIL promoter. Co-expression of p. 53 did not result in an increase in luciferase activity compared to the activity with the empty vector (FIG. 10B). These results further indicate that p53 does not regulate the SNAIL promoter.

The p53-independent regulation of SNAIL expression following DNA damage in vivo was examined. Wild-type and p53−/− mice were treated with 5 Gy of γ-radiation and expression of SNAIL in spleens was analyzed by Northern-blot (FIG. 10C). Six hours after irradiation, the expression of SNAIL was down-regulated in both, control and p53−/− mice. Therefore, SNAIL expression is similarly modulated in vivo following DNA damage. Overall, the above results demonstrated the requirement of a critical level of Snail for cancer development and indicate that failure to regulate Snail leads to cancer development in Combi-tTA-Snail mice.

Cross Between p53−/− Mice and combi-tTA-SNAIL Mice

Mice deficient in p53 (also termed “p53 null mice” or “p53−/− mice”) were crossed with combi-tTA-SNAIL mice, to yield Combi-tTA-SNAIL-p53−/− mice. It was found that these mice develop very large thymic lymphomas at an age of 2-3 months (FIG. 11). It was moreover found that these tumours infiltrated the lung, the heart, the mediastinal space and were essentially impossible to dissect. Micrographs of histological preparations of lymphoma, lung tumour and sebaceoma samples are shown in FIG. 12.

Surprisingly, it was found that Combi-tTA-SNAIL-p53−/− mice reproduced human cancers, also and in particular with respect to the dissemination and metastasis of malignant human cancers. Said Combi-tTA-SNAIL-p53−/− mice thus represent an ideal model to develop therapies targeting dissemination controls.

Discussion

The inventors have improved upon the single-plasmid system of Schultze et al., (1996) containing the regulating and expression elements of the original binary tetracycline system to allow induction and tight control of gene expression by tetracycline in mice to try to understand the relevance of Snail to human cancer development. In vitro studies have shown that Snail confers resistance to cell death induced by the withdrawal of survival factors (Vega et al, 2004). The physiological relevance of the Combi-tTA-Snail suppression was confirmed in vitro by assaying survival of Ba/F3 cells expressing Combi-tTA-Snail after IL-3 withdrawal. The analysis of the Snail-expressing mice identified that these mice develop cancer, mainly hematopoietic tumours. It is believed that the resistance to cell death conferred by Snail provides a selective advantage to cell migration important to cancer development (Vega et al, 2004). Thus, the hematopoietic cancers observed in the Combi-tTA-Snail mice demonstrate in vivo that transformation depends upon genetic changes that allow undifferentiated cells to grow outside their normal environment. Thus, these results provide evidence that Snail expression facilitates cell migration. The survival conferred by Snail, while reversible in vitro (FIG. 1), can escape such control in vivo.

In the mouse the Snail gene was previously implicated in the triggering of EMT, an important pathway to acquisition of the invasive phenotype in epithelial solid tumours (Batlle et al., 2000; Cano et al., 2000). The data obtained in connection with the present invention did not support this observation, as neither epithelial alterations nor non-invasive carcinomas developed in Combi-tTA-Snail mice. However, Combi-tTA-Snail mice expressed a limited amount of Snail. Thus, although present at a level sufficient to promote resistance to cell death elicited by growth factor withdrawal (FIG. 1), the transgene-encoded Snail may not be present at a level sufficient to alter EMT in Combi-tTA-SNAIL mice. This level of expression was, however, sufficient to induce cancer. It appears that Snail must be kept above a certain threshold level to achieve normal development. Consistent with this interpretation, Combi-tTA-Snail induced a tumorigenic but not migratory phenotype in MEFs. These findings indicate Snail does not require tumour formation before dissemination can place. However, these results cannot exclude a role for Snail in carcinoma development in a context where epithelial cells show or accumulate previous tumour alterations.

The inventors' results show that “increased” Snail expression induces cancer in mice with high frequency. These results suggest that Snail expression was protecting cells from death by genetic alterations as a consequence of an inherent, basal level of genetic instability. The inventors have, however, moreover demonstrated that Combi-tTA-Snail expression results in increased radioprotection. Thus, constitutive activation of Snail could confer radioresistance properties to the tumour-target cells. In concert with these results, the inventors show that both in vivo and in vitro Snail expression is modulated in response to DNA damage. However, although in vitro studies have suggested that aberrant expression of Snail alters the response to genotoxic stress by increasing p53 levels (Kajita et al., 2004), p53 response to DNA damage was not affected in Combi-tTA-Snail mice. The present inventors' results connect DNA damage with the requirement of a critical level of an EMT regulator for cancer development and it seems likely that failure to regulate Snail explains why Combi-tTA-Snail mice develop cancer. These findings further indicate that overexpression of Snail by human tumours could be of importance to cell fate selection by genotoxic anticancer agents.

Does the Snail-DNA damage interaction contribute to a physiologic defence mechanism exploited by human cancers? Snail is able to trigger EMT, an important pathway to acquisition of the invasive phenotype in epithelial solid tumours. Thus, under physiological conditions, DNA damage decreases Snail expression and could contribute to a transient inhibition of migratory capacity of tumour-target cell. With constitutive expression of Snail during transformation, this control is lost. Thus human cancers that overexpress Snail may have a survival advantage to genotoxic and potentially other forms of stress by exploiting physiologic mechanisms that evolved for the EMT, raising the possibility of strategies based on Snail for the treatment of human cancer.

### REFERENCES

Bathe, E., Sancho, E., Franci, C., Dominguez, D., Monfar, M., Baulida, J. and de Herreros, A. G. (2000) The transcription factor Snail is a repressor of E-cadherin gene expression in epithelial tumour cells. Nature Cell Biology 2: 84-89.
Cano, A., Pérez-Moreno, M. A., Rodrigo, I., Locascio, A., Blanco, M. J., del Barrio, M. G., Portillo, F., and Nieto, M. A. (2000) The transcription factor Snail controls epithelial-mesenchymal transitions by repressing E-cadherin expression. Nature Cell Biology 2: 76-83.
Kajita, M., McClinic, K. N., and Wade, P. A. (2004) Aberrant expression of the transcription factors snail and slug alters the response to genotoxic stress. Mo.l Cell. Biol. 24(17): 7559-7566.
Norris, P. S.; and Haas, M. (1997) A fluorescent p53GFP fusion protein facilitates its detection in mammalian cells while retaining the properties of wild-type p53. Oncogene 15(18): 2241-2247.
Palacios, R., and Steinmetz, M. (1985) IL-3 dependent mouse clones that express B-220 surface antigen, contain Ig genes in germ-line configuration, and generate B-lymphocytes in vivo. Cell 41:727-734.
Saito, T., Oda, Y., Kawaguchi, K., Sugimachi, K., Yamamoto, H., Tateishi, N., Tanaka, K., Matsuda, S., Iwamoto, Y., Ladanyi M, et al. (2004) E-cadherin mutation and Snail overexpression as alternative mechanisms of E-cadherin inactivation in synovial sarcoma. Oncogene 23(53): 8629-8638.
Schultze, N., Burki, Y., Lang, Y., Certa, U., and Bluethmann, H. (1996) Efficient control of gene expression by single step integration of the tetracycline system in transgenic mice. Nature Biotechnology 14: 499-503.
Sugimachi, K., Tanaka, S., Kameyama, T., Taguchi, K., Aishima, S., Shimada, M., Sugimachi, K., and Tsuneyoshi, M. (2003) Transcriptional repressor snail and progression of human hepatocellular carcinoma. Clin. Cancer Res. 9(7): 2657-2664.
Vega, S., Morales, A. V., Ocana, O. H., Valdes, F., Fabregat, I., and Nieto, M. A. (2004) Snail blocks the cell cycle and confers resistance to cell death. Genes Dev. 18(10):1131-1143.

1. A transgenic non-human mammal comprising in its genome a transgene that comprises a nucleic acid sequence encoding the SNAIL protein

2. The transgenic non-human mammal of claim 1, wherein the expression of said transgene is exogenously regulated by an effector substance.

3. Transgenic non-human mammal according to claim 1, wherein said mammal is a rodent.

4. Transgenic non-human mammal according to claim 3, wherein said rodent is a mouse or a rat.

5. Transgenic non-human mammal according to claim 1, wherein said mammal suffers from an epithelial and/or mesenchymal tumour and/or cancer.

6. Transgenic non-human mammal according to claim 1, obtainable by crossing a non-human mammal with another non-human mammal carrying a mutation in the gene encoding the p53 protein.

7. Transgenic non-human mammal according to claim 1, further characterised in that said non-human mammal carries a mutation in the gene encoding the p53 protein.

8. Transgenic non-human mammal according to claim 1, further characterised by a homozygous p53 null mutation.

9. The progeny of a transgenic non-human mammal according claim 1.

10. A primary cell or tissue sample which is derived from the transgenic non-human mammal according to claim 1.

11. A cell line comprising in its genome a transgene, wherein said transgene is characterised as in claim 1.

12. A cell line which is obtainable from the transgenic non-human mammal, its progeny, or the primary cell or tissue sample according to claim 1.

13. (canceled)

14. A method for screening, searching, identifying, validating, optimizing, discovering, developing and/or evaluating compounds for the prevention and/or treatment of a mesenchymal or epithelial tumour or a DNA damage-based disease or for repositioning known drugs or combinations of compounds, which comprises administering a candidate compound to a transgenic non-human mammal according to claim 1, and monitoring the response.

15. A method for identifying a compound which inhibits or reduces the level of expression of SNAIL gene or its expression products, which comprises administering to a transgenic non-human mammal according to claim 1, wherein the level of SNAIL gene or its expression products in a tissue is known, with a candidate compound, and, subsequently, quantifying the level of SNAIL gene or its expression products in said tissue, and selecting a compound which is able to reduce the known level of SNAIL gene or its expression products.

16. A method for screening, searching, identifying, discovering, developing and/or evaluating compounds for the prevention and/or treatment of a mesenchymal or epithelial tumour or DNA damage-based disease or for repositioning known drugs or combinations of compounds, which comprises contacting a cell line, or a primary cell, or a tissue sample according to claim 10, and monitoring the response.

17. A method for identifying a compound which inhibits or reduces the level of expression of SNAIL gene or its expression products which comprises contacting a candidate compound contacted with a cell line, or with a primary cell, or with a tissue sample according to claim 10, wherein the level of SNAIL expression products in said cell line, primary line or tissue sample is known; subsequently, quantifying the level of SNAIL expression products in said tissue, and selecting a compound which is able to inhibit or reduce the level of SNAIL expression products.

18. Use of a compound which inhibits or decreases the levels of the SNAIL expression products or reverts the effects of an increased level of SNAIL expression products in the manufacture of a pharmaceutical composition for prevention and/or treatment of a tumour selected from an epithelial tumour or a mesenchymal tumour, or of a DNA damage-based disease.

19. Use according to claim 18, wherein said compound is selected from the group consisting of antisense SNAIL mRNA, ribozymes, triple helix molecules, small interference RNA (siRNA), antibodies anti-SNAIL, enzymes or proteins which regulate the activity of SNAIL protein, and mixtures thereof.

20. A pharmaceutical composition comprising a therapeutically effective amount of a compound which inhibits or decreases the levels of the SNAIL expression products or reverts the effects of an increased level of SNAIL expression products together with one or more pharmaceutically acceptable excipients and/or carriers.

21. Pharmaceutical composition according to claim 20, which comprises a vector comprising a compound which inhibits or decreases the levels of the SNAIL expression products.

22. Pharmaceutical composition according to claim 21, wherein said compound which inhibits or decreases the levels of the SNAIL expression products is selected from the group consisting of antisense SNAIL mRNA, ribozymes, triple helix molecules, small interference RNA (siRNA), antibodies anti-SNAIL, enzymes or proteins which regulate the activity of SNAIL protein, and mixtures thereof.

23. A kit for determining the invasive and/or metastatic capacity of an epithelial or mesenchymal tumour, or the local growth capacity of an epithelial or mesenchymal tumour, or for in vitro diagnosing a condition in a subject, said condition being selected from an epithelial tumour, a mesenchymal tumour and a DNA damage-based disease, or for determining the stage or severity of said condition in a subject, or for determining the predisposition of a subject to develop said condition, or for monitoring the effect of the therapy administered to a subject with said condition, or for screening, searching, identifying, discovering, developing and/or evaluating compounds for the prevention and/or treatment of a mesenchymal or epithelial tumour or DNA damage-based disease which comprises an antibody that specifically recognizes SNAIL protein in a suitable packing, or primer pair designed to specifically amplify a nucleic acid having a sequence that is specific to SNAIL.
